(19) United States
(12) Patent Application Publication
(10) Pub. No.: US 2020/0294677 A1
(43) Pub. Date: Sep. 17, 2020

(54) SYSTEMS AND METHODS FOR IMPORTING AND ANALYZING MEDICAL, WELLNESS, AND/OR TRAINING DATA AND GENERATING INTERACTIVE COMPUTER DISPLAYS

(71) Applicant: Empericus, LLC, Redwood City, CA (US)

(72) Inventors: Nuno F. M. Godinho, Reading (GB); Ronald R. Pruitt, Redwood City, CA (US); David C. White, Beach Haven, NJ (US); Robert D. Mack, Park Ridge, NJ (US); Philip C. Sproger, Center Valley, PA (US)

(21) Appl. No.: 16/816,801

(22) Filed: Mar. 12, 2020

Related U.S. Application Data
(60) Provisional application No. 62/818,488, filed on Mar. 14, 2019.

(51) Int. Cl. G16H 50/50; G16H 10/60; G16H 15/00; G16H 50/30; G16H 50/20; G06F 40/40

(52) U.S. Cl. CPC G16H 50/50; G16H 10/60; G16H 15/00; G06F 3/04847; G16H 50/20; G06F 40/40; G16H 50/30

(57) ABSTRACT

The present disclosure relates to systems and methods for importing medical, wellness, and/or training/performance data for a subject. Natural language processing (NLP) may be implemented on the imported records to identify and extract keywords. A machine learning (ML) system determine information for an imported record. The information may include, but is not limited to a type (e.g., medical, wellness, or training/performance) of an imported record, an affected body system, an affected body part, a severity/impact (e.g., severity of harm or quality), and an indication whether a condition associated with the imported record is currently active/in-treatment or not. In addition, one or more interactive computer-generated displays including elements may be generated utilizing the particular information determined from the imported records, wherein the one or more interactive computer-generated displays provide pictorial representation of significant events in a subject's medical, wellness, and/or training/performance condition over time.

### CROSS-REFERENCE TO RELATED APPLICATIONS

The present application claims the benefit of U.S. Provisional Patent Application Ser. No. 62/818,488, filed Mar. 14, 2019, by Nuno F. M. Godinho et al., for SYSTEMS AND METHODS FOR IMPORTING AND ANALYZING MEDICAL, WELLNESS, AND/OR TRAINING DATA AND GENERATING INTERACTIVE COMPUTER DISPLAYS, which is hereby incorporated by reference in its entirety.

### BACKGROUND

### Technical Field

The present disclosure relates to systems and methods for applying natural language processing and machine learning to medical, wellness, and/or training/performance data to generate interactive computer-generated displays of a subject's health, wellness, and/or training/performance over time.

### Background Information

Typically, a subject's medical, wellness, training/performance data may be expansive, may cover a long period of time, and may be stored in a variety of formats at many different locations. Furthermore, only a portion of this expansive data set may relate to a given injury or condition. As such, it can be difficult and time-consuming for medical professionals, athletic trainers, and others to sift through this data set to access and understand the portion that is of importance to them. Accordingly, a need exists for a system and method that can assist users in filtering a subject's medical, wellness, and/or training/performance data to identify portions relating to particular events and/or conditions.

### SUMMARY

Briefly, the present disclosure relates to systems and methods for importing medical, wellness, and/or training/performance data for a subject (human being, animal, plant, vegetation, etc.), implementing natural language processing (NLP) on imported data (i.e., imported records) to identify and extract keywords, applying machine learning to determine information from the imported records, and generating interactive computer displays having elements representing significant events or conditions determined for the subject. The elements included on the interactive computer displays may provide summary information of the significant events or conditions and links for accessing increasingly greater levels of detailed information concerning those events or conditions. Specifically, a medical, wellness, and training (MWT) system may import data (e.g., one or more medical or other records) from one or more data sources and/or one or more end user devices. The imported records may include a structured data portion (e.g., codes) and an unstructured data portion (e.g., free text clinician notes, commentary, etc.). The data sources may include, but are not limited to, an electronic medical record system (EMRS), an electronic medical device, a health device, a wellness device, a training device, and/or a performance device. In addition or alternatively, the data may be manually provided from an end user device to the MWT system.

A natural language processor (NLP) unit of the MWT system may analyze the imported records and extract one or more keywords from the imported records. For each imported record, the NLP unit may analyze the structured data portion to derive a context for the unstructured data portion of the same imported record, e.g., based on one or more rules. For example, the structured data portion of a given imported record may include one or more codes for particular clinical findings, symptoms, diagnoses, procedures, body structures, organisms and other etiologies, substances, pharmaceuticals, devices and specimens. The NLP unit may detect such codes and apply the one or more rules to derive a context for the unstructured data portion of the given imported record. Based on the derived context, the NLP unit may identify one or more keywords, and may search the unstructured data portion of the given imported record for occurrences of the one or more keywords. In some embodiments, the NLP unit may analyze the unstructured data portion of the imported record to derive a context of the structured data portion and populate one or more fields of the structured data portion with one or more codes or identifiers.

A machine learning engine of the MWT system may determine MWT information for an imported record. The MWT information may include, but is not limited to, a type of record (e.g., medical, wellness, or training/performance), an affected body system, an affected body part, a severity/impact (e.g., severity of harm or quality), and an indication whether a condition associated with the imported record is currently active/in-treatment or not. Inputs to the machine learning engine may include the keywords obtained by the NLP unit, the imported records or one or more portions thereof, and weights for elements in the imported records. The MWT system, and specifically the machine learning engine, may create an MWT record and store the MWT information in the created MWT record with additional information, such as, but not limited to, a date associated with the imported record. In addition, the MWT information from the MWT records may be indexed such that medical professionals, athletic trainers, and other users may utilize one or more user interfaces to search the stored MWT records to, for example, identify subjects with particular conditions, symptoms, severity of harm, etc.

In some embodiments, the machine learning engine may be implemented as a supervised machine learning system. For example, training data that includes imported records, extracted keywords, predetermined weights assigned to elements in the imported records, output records, and/or the indexed data from the MWT records may be used to train the MWT system.

The MWT system may generate one or more interactive computer displays. The interactive computer displays may include a lifeline for each type of data (e.g., medical, wellness, or training/performance) included in the MWT records. The lifelines may be a longitudinal, vertical, diagonal, or any shaped graphs and may be configured to present a particular time interval (e.g., days, months, or years). The MWT system may, for the time interval of the lifeline, aggregate (e.g., group together) MWT records whose determined date falls within the time interval of the lifeline into elements on the lifeline. The MWT system may, for each time interval, include or omit an element (e.g., marking) from the lifeline and present one or more graphical affordances (e.g., size, color, animation, etc.) for the element based on, for example, the severity/impact and/or or other information determined for the one or more MWT records included in the aggregate.

In response to the selection of an element on a lifeline, e.g., by a user, the MWT system may create and present a listing of the aggregated MWT records associated with the selected element. In response to the selection of a given entry from the list, the MWT system may retrieve and present a summary of the MWT record associated with the given entry. The summary, for example, may be created by the MWT system and included in the MWT record. In response to a request for additional detail, e.g., from the user, the MWT system may retrieve and present the imported record or one or more portions thereof. A user interface element, such as a slider, may be associated with the lifeline to adjust the time period being represented, thereby allowing the user to select one or more time periods of interest. As such, the lifeline, generated by the MWT system, provides a pictorial representation, such as a linear representation, of significant events in a subject's medical, wellness, and/or training/performance condition over time. A lifeline may also indicate periods of time where events having a high severity/impact have occurred, e.g., through highlighted. In addition, a user may navigate through a subject's medical, wellness, and/or training/performance data by interacting with the elements presented on the lifeline.

The MWT system may utilize the MWT records to highlight one or more body parts on an interactive computer-generated body map, which may depict at least a portion of the human anatomy. For example, the MWT system may highlight body parts for which data exists. In some embodiments, different graphical affordances may be used to represent different types of records (e.g., medical, wellness, and/or training/performance). The MWT system may aggregate MWT records that involve the same body part and are of the same type (e.g., medical, wellness, or training/performance). The MWT system may then apply one or more graphical affordances (e.g., shading, color, etc.) to the body part associated with the aggregate of MWT records that belong to the same type of record. As such, the MWT system may provide a visual indication on the body map indicating the type of record(s) associated with that body part and the number of aggregated MWT records. Advantageously, the graphical affordances applied to body parts on the interactive computer-generated body map allow a user to quickly and easily identify which body parts have associated medical, wellness, and/or training/performance data (e.g., MWT records).

The MWT system may also apply one or more graphical affordances to the body parts to indicate a severity level (e.g., healthy, mild, moderate, severe). Specifically, the MWT system may apply one or more graphical affordances (e.g., shading, color, etc.) to the body part associated with an aggregate of MWT records based on the severity and/or other information included in the MWT records of the aggregate (e.g., whether a condition is active or not as indicated in an MWT record). As such, the MWT system may highlight body parts based on the severity level indicated in the MWT records determined for the body parts. Advantageously, the graphical affordances applied to body parts indicating severity of harm or condition on the interactive computer-generated body map allow a user to quickly and easily identify those body parts that may have problems and/or associated harm. In response to the selection of a body part on the body map, the MWT system may create and present a listing of the aggregated MWT records associated with the selected body part. In response to the selection of a given entry from the list, the MWT system may retrieve and present a summary of the MWT record associated with the given entry. The summary, for example, may be included in the MWT record when it is created by the MWT system. In response to a request for additional detail, e.g., from the user, the MWT system may also retrieve and present the imported record. In addition, the MWT system may include one or more body system filters associated with the body map. In response to the selection of a particular body system filter, the MWT system may only utilize the MWT records associated with the selected body system when highlighting body parts on the body map. Like the lifeline, the body map also may provide a pictorial representation of a subject's medical, wellness, and/or training/performance condition. However, with the body map, this information may be presented in terms of human anatomy, where areas of interest, e.g., body parts, are emphasized, e.g., highlighted, as a function of information from the imported records. In addition, a user may navigate through medical, wellness, and/or training/performance data by interacting with the body map, and may utilize the body map to focus the user's search on body parts and/or body systems of interest. In other words, whereas the lifeline provides a time-centric pictorial representation of the subject's medical, wellness, and/or training/performance condition, the body map provides a body area/part centric representation. Providing both may allow a user to make a quick determination as to which body parts may be problematic and for how long or how recently.

Further, the MWT system may link the body map to the lifeline. For example, a user may utilize the slider or other mechanism to focus the lifeline on a particular time period. In response, the MWT system may filter the MWT records to those corresponding to that time period and use the filtered MWT records to mark the body parts of the body map.

It should be understood that a variety of additional features and alternative embodiments may be implemented other than those discussed in this Summary. This Summary is intended simply as a brief introduction to the reader and does not indicate or imply that the examples mentioned herein cover all aspects of the present disclosure, or are necessary or essential aspects of the disclosure.

In particular, the subject-matter according to the present invention may also be considered to comprise the following aspects 1 to 20:

1. A system, comprising:

a processor when executed configured to:
extract, based on an implementation of natural language processing, one or more keywords from each of a plurality of imported records associated with a subject,
determine, utilizing the extract keywords and the imported records, information for each of the imported records, wherein the information includes at least a type associated with the imported record, an affected body system associated with the imported record, an affected body part associated with the imported record, an impact level associated with the imported record, and an indication whether a condition, associated with the imported record, is active or not;
generate, utilizing the information determined for each of the plurality of imported records for the subject, one or more interactive computer-generated displays that provide a pictorial representation of the subject's medical history, wellness, training condition, and/or performance condition over time.
   2. The system according to aspect 1, wherein the plurality of imported of records includes at least one of medical records including medical data, wellness records including wellness data, and performance records including training and/or performance data.
   3. The system according to aspect 1 or 2, wherein the plurality of imported record includes structured data and unstructured data, and the structured data includes one or more codes that are associated with at least one of System Nomenclature of Medicine-Clinical Terms and International Classification of Diseases, Tenth Revision, Clinical Modification.
   4. The system according to one of the preceding aspects, wherein the subject is one of a human being, an animal, plant, or vegetation.
   5. The system according to one of the preceding aspects, wherein the processor is further configured to:
generate a medical interactive computer-generated lifeline for the subject utilizing the information determined for medical imported records of the subject, wherein one or more markings are included at one or more positions on the medical interactive computer-generated lifeline to represent a severity of harm, and each position is associated with a different time interval on the medical interactive computer-generated lifeline.
   6. The system according to aspect 5, wherein the one or more markings include a first marking that is a first size and at a first time interval on the medical interactive computer-generated lifeline and a second marking that is a second size and at a second time interval on the medical interactive computer-generated lifeline, and wherein a first severity of harm represented by the first marking is different than a second severity of harm represented by the second marking.
   7. The system according to aspect 6, wherein the processor is further configured to:

display, in response to receiving a selection of the first marking, a listing of entries corresponding to the medical imported records having a date that falls within first time interval.

8. The system according to aspect 7, wherein the processor is further configured to:

display, in response to receiving a selection a of a particular entry in the listing, selected information associated with the particular entry, wherein the particular entry corresponds to a particular medical imported record having the date that falls within the time interval.

9. The system according to one of the preceding aspects, wherein the processor is further configured to:

generate a wellness interactive computer-generated lifeline for the subject utilizing the information determined for wellness imported records of the subject, wherein one or more markings are included at one or more positions on the wellness interactive computer-generated lifeline to represent a wellness quality, and each position is associated with a different time interval on the wellness interactive computer-generated lifeline.

10. The system according to aspect 9, wherein the one or more markings include a first marking that is a first size and at a first time interval on the wellness interactive computer-generated lifeline and a second marking that is a second size and at a second time interval on the wellness interactive computer-generated lifeline, and wherein a first wellness quality represented by the first marking is different than a second wellness quality represented by the second marking.
11. The system according to one of the preceding aspects, wherein the processor is further configured to:

generate a training and/or performance interactive computer-generated lifeline for the subject utilizing the information determined for training and/or performance imported records of the subject, wherein one or more markings are included at one or more positions on the training and/or performance interactive computer-generated lifeline to represent a training and/or performance quality, and each position is associated with a different time interval on the training and/or performance interactive computer-generated lifeline.

12. The system according to aspect 11, wherein the one or more markings include a first marking that is a first size and at a first time interval on the training and/or performance interactive computer-generated lifeline and a second marking that is a second size and at a second time interval on the training and/or performance interactive computer-generated lifeline, and wherein a first training and/or performance quality represented by the first marking is different than a second training and/or performance quality represented by the second marking.
13. The system according to one of the preceding aspects, wherein the processor is further configured to:

generate an interactive computer-generated body map for the subject utilizing the particular determined for each of the plurality of imported records for the subject,

wherein one or more body parts on the interactive computer-generated body map are highlighted to indicate severity of harm body parts based on at least the first impact identifier of the information determined for medical imported records of the subject.

14. The system according to one of the preceding aspects, wherein the processor is further configured to:

generate an interactive computer-generated body map for the subject utilizing the information determined for the imported records of the subject,
wherein the one or more body parts on the interactive computer-generated body map are highlighted, utilizing the information determined for the imported records of the subject, to indicate which of the one or more body parts are associated with medical data, wellness data, or training and/or performance data.
   15. A system, comprising:

a processor when executed configured to:
is import a plurality of records, for a subject, from a data source and/or an end user device;
create, based on natural language processing and machine learning, an output record for each of the plurality of imported records, where each output record includes a type identifier for a type of the imported record, a body system identifier for an affected body system, a body part identifier for an affected body part, an impact identifier for an impact level, an is-active identifier for whether a condition is active or not, and a date identifier identifying a particular date;
create a first aggregate for a first set of the records, wherein each output record of the first aggregate includes the date identifier identifying the particular date that falls within a first time interval;
create a second aggregate for a second set of the records, wherein each output record of the second aggregate includes the date identifier identifying the particular date that within a second time interval; and
generate an interactive computer-generated lifeline for the subject that provides a pictorial representation of the user's medical history, wellness, or training over time,
   wherein a first marking is included at a first position, corresponding to the first time interval, on interactive computer-generated lifeline to represent the first aggregate, and
   wherein a second marking is included at a second position, corresponding to the second time interval, on the interactive computer-generated lifeline to represent the second aggregate.
      16. The system according to aspect 15, wherein the processor when executed is further configured to:

display, in response to receiving a selection of the first marking, a listing of entries, where each entry is associated with a different record of the first set of records that make up the first aggregate; and

display, in response to receiving a selection of a particular entry, information associated with a particular record of the first set of records.

17. The system according to aspect 15 or 16, wherein the first marking is a first size and represents a first severity of harm, and the second marking is a second size and represents a second severity of harm that is different than the first severity of harm.
18. A system, comprising:

a processor when executed configured to:
import a plurality of records, for a subject, from a source and/or an end user device;
create, based on natural language processing and machine learning, an output record for each of the plurality of imported records, where each output record includes a body system identifier for an affected body system, a body part identifier for an affected body part, an impact identifier for an impact level, an is-active identifier for whether a condition is active or not, and a date identifier identifying a particular date;
create a first aggregate for a first set of the output records, wherein each output record of the first aggregate includes a first same body part identifier, assigned to a first body part, for the affected body part;
create a second aggregate for a second set of the output records, wherein each output record of the second aggregate includes a second same body part identifier, assigned to a second body part, for the affected body part;
generate an interactive computer-generated body map for the user, the interactive computer-generated body map including at least a front view of a human avatar and a back view of the human avatar, wherein the front view and the back view include a pictorial representation of a human anatomy having body parts;
highlight the first body part on the interactive computer-generated body map with a first affordance indicating a first severity of harm, wherein the first severity of harm for the first body part is determined based on at least the impact identifier for the impact level included in the first set of records that make up the first aggregate; and
highlight the second body part on the interactive computer-generated body map with a second affordance indicating a second severity of harm, wherein the second severity of harm for the second body part is determined based on at least the impact identifier included in the second set of output records that make up the second aggregate.
   19. The system according to aspect 18, wherein the processor when executed is further configured to:
display, in response to receiving a selection of the first body part, a listing of entries associated with the first set of output records that make up the first aggregate; and display, in response to receiving a selection of a particular entry form the listing, information associated with a particular record of the first set of records.
   20. The system according to aspect 18 or 19, wherein the records include medical records.

### BRIEF DESCRIPTION OF THE DRAWINGS

The description below refers to the accompanying drawings, of which:

FIG. 1 is a high-level functional block diagram of a system in accordance with one or more embodiments described herein;

FIG. 2 is a schematic illustration of an example ID index in accordance with one or more embodiments described herein;

FIG. 3A is a schematic illustration of an example header for one or more MWT records in accordance with one or more embodiments described herein;

FIGS. 3B-3E are schematic illustrations of example MWT records in accordance with one or more embodiments described herein;

FIG. 4 is a schematic illustration of s flow diagram of an example method for creating an MWT record in accordance with one or more embodiments described herein;

FIG. 5 is a schematic illustration of an example computer display depicting interactive computer-generated lifelines in accordance with one or more embodiments described herein;

FIG. 6 is a schematic illustration of an example listing associated with the MWT records of an aggregate represented by a marking on an interactive computer-generated lifeline in accordance with one or more embodiments described herein;

FIG. 7 is a schematic illustration of an example entry from a listing in accordance with one or more embodiments described herein;

FIG. 8 is a schematic illustration of an example computer display depicting an interactive computer-generated body map that highlights body parts of a human being based on data type in accordance with one or more embodiment described herein;

FIG. 9 is a schematic illustration of an example listing associated with the MWT records of an aggregate represented by a body part on the interactive computer-generated body map of a human being in accordance with the one or more embodiments described herein;

FIG. 10 is a schematic illustration of an entry from a listing associated with MWT records for a human being in accordance with one or more embodiments described herein;

FIG. 11 is a schematic illustration of a computer display depicting an interactive computer-generated body map that highlights body parts of a human being based on severity of harm in accordance with one or more embodiment described herein;

FIG. 12 is a schematic illustration of an example computer display depicting an interactive computer-generated body map that highlights body parts of an animal based on data type in accordance with one or more embodiment described herein;

FIG. 13 is a schematic illustration of an example listing associated with the MWT records of an aggregate represented by a body part on the interactive computer-generated body map of an animal in accordance with the one or more embodiments described herein;

FIG. 14 is a schematic illustration of an entry from a listing associated with MWT records for an animal in accordance with one or more embodiments described herein;

FIG. 15 is a schematic illustration of a computer display depicting an interactive computer-generated body map that highlights body parts of an animal based on severity of harm in accordance with one or more embodiment described herein;

FIG. 16 is a schematic illustration of an example computer display depicting an interactive computer-generated body map that highlights body parts of a plant based on data type in accordance with one or more embodiment described herein;

FIG. 17 is a schematic illustration of an example listing associated with the MWT records of an aggregate represented by a body part on the interactive computer-generated body map of a plant in accordance with the one or more embodiments described herein;

FIG. 18 is a schematic illustration of an entry from a listing associated with MWT records for a plant in accordance with one or more embodiments described herein;

FIG. 19 is a schematic illustration of a computer display depicting an interactive computer-generated body map that highlights body parts of a plant based on severity of harm in accordance with one or more embodiment described herein;

FIG. 20 is a schematic illustration of a mapping in accordance with one or more embodiments described herein;

FIG. 21 is a schematic illustration of a flow diagram of an example method for generating and operating an interactive computer-generated lifeline according to one or more embodiments described herein; and

FIG. 22 is a schematic illustration of a flow diagram of an example method for generating and operating an interactive computer-generated body map according to one or more embodiments described herein.

### DETAILED DESCRIPTION OF AN ILLUSTRATIVE EMBODIMENT

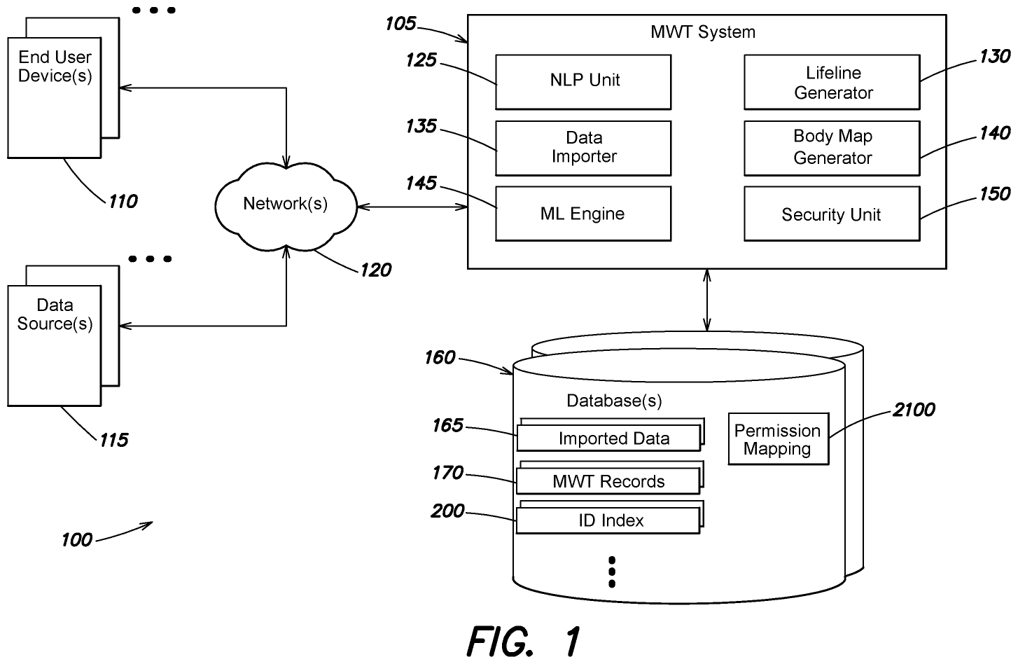

FIG. 1 is a high-level, functional block diagram of an example system 100 in accordance with one or more embodiments. The system 100 may include a medical, wellness, and training/performance (MWT) system 105, one or more end user devices 110, and one or more data sources 115. The MWT system 105, end user devices 110, and data sources 115 may communicate over one or more data communication networks 120.

The MWT system 105 may include a natural language processing (NLP) unit 125, a lifeline generator 130, a data importer 135, a body map generator 140, a machine learning (ML) engine 145, and a security unit 150.

The MWT system 105 may be coupled to one or more databases 160 configured to store medical, wellness, and/or training/performance records (imported data) 165 imported from the one or more data sources 115 and/or end user devices 110, MWT records 170 generated for the imported records, an ID index 200, a permissions mapping 2100, and one/or more other data structure according to the one or more embodiments described herein.

The data sources 115 may be systems and/or devices from which the MWT system 105 may import medical, wellness, and/or training/performance data. For example, the one or more data sources 115 may include, but are not limited to, an electronic medical record system (EMRS) of a hospital, physician's practice, or other medical facility. Other exemplary data sources 115 include an electronic medical device, a health device, a wellness device, a training device, and/or a performance device (e.g., Catapult, StatSports, Apple Watch, FitBit, Garmin, Whoop and Polar, etc.).

The one or more end user devices 110 may include, but are not limited to, a computer terminal or workstation, tablet, smartphone, virtual reality (VR) systems, augmented reality (AR) systems, or other types of computing devices having a display. As described herein, the MWT system 105 may present one or more interactive computer displays at the end user devices 110.

In some embodiments, one or more of the data importer 135, NLP unit 125, ML engine 145, lifeline generator 130, and/or body map generator 140 may be implemented through one or more software modules or libraries containing program instructions that perform the methods described herein, among other methods. The software modules may be stored in one or more memories, such as a main memory, a persistent memory, and/or a computer readable media, of a data processing device, and may be executed by one or more processors. Other computer readable media may also be used to store and execute these program instructions, such as one or more non-transitory computer readable media, including optical, magnetic, or magneto-optical media. In other embodiments, one or more of the data importer 135, NLP unit 125, ML engine 145, lifeline generator 130, and/or body map generator 140 may be implemented in hardware, for example through hardware registers and combinational logic configured and arranged to produce sequential logic circuits that implement the methods described herein. In other embodiments, various combinations of software and hardware, including firmware, may be utilized to implement the systems and methods of the present disclosure.

The security unit 150 may establish credentials for users of the MWT system 105. A user (e.g., a subject or a user interested in a subject's data) may utilize the end user device 110 to establish a unique account with the MWT system 105. Specifically, a user may utilize an end user device 110 to access one or more user interfaces (UIs), such as webpages, associated with the MWT system 105 and generated by the data importer 135, and then utilize the client-facing UI s to provide personal information (e.g., name, date of birth, address, and/or social security number, etc.). The security unit 150 of the MWT system 105 may utilize the personal information to establish a unique MWT system account for the user (e.g., register the user with the MWT system 105). Subsequently, the user may provide user credentials (e.g., username and password) to the MWT system 105, and the MWT system 105 may provide the user with access to the user's unique MWT system account. As such, the MWT system 105 may then provide the user with access to a subject's data maintained by the MWT system 105 and the features and functions, such as the interactive computer-generated displays, provided by the MWT system 105. In addition or alternatively, and based on a permissions mapping 2100 stored in DBs 160, a user may grant access to at least a portion of the subject's data in the MWT system 105 to others, e.g., medical providers, trainers, etc. For example, the user may grant access to his or her own data. The security unit 150 may create an ID index for each subject. As such, the MWT system 105 may provide the user with access to the features and functions, such as the interactive computer displays, generated by the MWT system 105 utilizing MWT records storing data associated with one or more subjects.

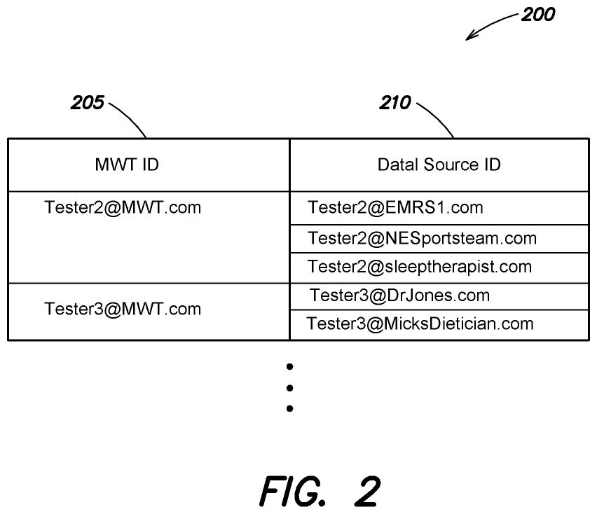

FIG. 2 is a schematic illustration of an example ID index 200 in accordance with one or more embodiments. The ID index 200 may store credentials for accessing a subject's information from one or more of the data sources 115. For example, the ID index 200 may store associations between user identifiers, e.g., MWT system identifiers, assigned to users registered with the MWT system 105 and one or more data source identifiers assigned to the subjects by one or more of the data sources 115. Specifically, ID index 200 may include a first section 205, such as a column or a row, which stores the MWT system identifiers. In addition, ID index 200 may include a second section 210, such a column or row, which stores data source identifiers assigned to subjects by data sources 115.

As described, a user may grant access to some or all of the user's information to one or more other users of the MWT system. As an example, a user (who is also a subject) may grant access to a first portion of the user's information to a given medical provider and another portion to a given athletic trainer.

A registered user may utilize an end user device 110 to access one or more UIs associated with the MWT system 105, and provide user credentials (e.g., user identifier stored in first section 205 and password) to the MWT system 105. The MWT system 105 may then provide the user with access to the user's MWT system account. The MWT system 105 may receive the data source identifiers over the network 120 from the end user device 110, e.g., inputted by a user. The MWT system 105 may then store the user's MWT system identifier in the first section 205 of the ID index 200, and store the user's one or more data source identifiers in second section 210 of the ID index 200. As such, the ID index 200 may be utilized by the MWT system 105 to link data imported from one or more of the data sources 115 to a user's MWT system identifier. The MWT system 105 may store the ID index 200 in one or more or DBs 160.

For example, and as depicted in FIG. 2, a first user registered with the MWT system 105 has a user identifier of “Tester2@MWT.com” that is stored by the MWT system 105 in first section 205. In addition, the user has three data source identifiers “Tester2@EMRS1.com”, “Tester2@NESportsteam.com”, and “Tester2@Sleeptherapist.com” that respectively correspond to data sources EMRS1, NESportsteam, and Sleeptherapist, and that are stored by the MWT system 105 in the second section 210. EMRS1 may be a server/DB that stores electronic medical records for a plurality of users. NESportsteam may be a server/DB that stores electronic training/performance, and/or medical information for a plurality of athletes that play for NESportsteam. Further, Sleeptherapist may be a server/DB that stores electronic sleep information (i.e., wellness data) for a plurality of users. As such, and when records are imported from these data sources for Tester2, the MWT system 105 may link the imported records to user identifier of “Tester2@MWT.com”. The MWT system may then store the imported records on the one or more DBs 160 with the identifier of “Tester2@MWT.com.”

Similarly, and as depicted in FIG. 2, a second user registered with the MWT system 105 has a user identifier of “Tester3 @MWT.com” that is stored by the MWT system 105 in first section 205. In addition, the user has two data source identifiers “Tester3@DrJones.com” and “Tester3@MicksDietician.com” that respectively correspond to data sources DrJones and MicksDietician, and that are stored by the MWT system 105 in the second section 210. DrJones may be a server/DB that stores electronic medical records for patients of Dr. Jones. MicksDietician may store electronic diet information (e.g., wellness data) for users that seek dietary assistance from Mick, a personal dietician. As such, and when records are imported from these data sources for Tester3, the MWT system 105 may link the imported records to user identifier of “Tester3 @MWT.com”. The MWT system 105 may then store the imported records on the one or more DBs 160 with the identifier of “Tester3@MWT.com.”

In some embodiments, a user may have to provide authorization to one or more of the data sources 115 to allow it to provide imported records to the MWT system 105.

The structure of the ID index 200 and the values and information stored in the first section 205 and second section 210 are for illustrative purposes only. It is expressly contemplated that the ID index 200 may have different structures and may include less, additional, or other information such that an association between a user's MWT system identifier and the subject's data source identifiers is maintained/stored.

Communication between the MWT system 105 and the data sources 115 and/or end user devices 110 may be by Secure Socket Layer (SSL) connections or any of a variety of different types of connections that enhance security of the connection and the encryption of the data being sent over the connection.

In an embodiment, the data source 115 is an EMRS that supports the Health Level-7 (HL7) interface standards. The MWT system 105 may establish a secure virtual private network (VPN) connection to the HL7 compatible EMRS. The MWT system 105 may receive one or more HL7 messages (e.g., pushed or pulled) from the EMRS, and the data importer 135 may parse the HL7 messages to determine if the information in the HL7 messages is related to a user registered with the MWT system 105.

For example, the data importer 135 may analyze the HL7 messages to extract a data source identifier from the HL7 messages. The data importer 135 may then determine if the extracted data source identifier is stored in the second section 210 of the ID index 200. If the data source identifier is stored in the ID index 200, then the data importer 135 may determine that the user is registered with the MWT system 105 (the user's MWT system identifier is the value stored in the corresponding first section 205 of the ID index 200). If the user is registered with the MWT system 105, the data importer 135 may send an acknowledgment message to the EMRS, and the EMRS may extract medical data, convert the extracted medical data into a Fast Healthcare Interoperability Resources (FHIR) bundle, and transmit the FHIR bundle to the MWT system 105. The data importer 135 may then store the FHIR bundle on the one or more DBs 160 with the user identifier and/or the data source identifier utilizing ID index 200.

In an embodiment, the data source 115 is an EMRS that supports FHIR or other application program interface (API) based integration. Under this circumstance, the MWT system 105 may establish a secure VPN connection with the EMRS that supports FHIR or the other API based integration. The data importer 135 may create one or more subscriptions to monitor for any additions, changes, or deletions made to EMRS using FHIR standards and the ID index 200. For example, whenever a subscription is active for the user identifier and/or the data source identifier in the ID index 200, additions, changes, and deletions may be pushed to the MWT system 105. The data importer 135 may store the additions, changes, and deletions as FHIR bundles on the one or more DBs 160 with the user identifier and/or the data source identifier utilizing ID index 200.

In an embodiment, the MWT system 105 may be unable to import data from the data source or the data source may not support HL7, FHIR, or other API based integrations. Under this circumstance, the user may request, from the data source 115, a digital version of the user's medical, wellness, and/or training/performance record that is in any of a variety of different formats. Such formats may include, but are not limited to, Continuity of Care Document (CCD), Clinical Data Repository (CDR), Clinical Document Architecture (CDA), Consolidated CDA (C-CDA), comma-separated values (CSV), etc. The user may then utilize an end user device 110 to gain access to his/her account, and then utilize one or more client-facing interfaces to upload the digital version from the end user device 110. The MWT system 105 may then receive the digital version from the end user device 110 via network 120, such that the data is imported by the MWT system 105. The data importer 135 may then generate an FHIR bundle for all data in the uploaded file that can be successfully parsed by the data importer 135. The data importer 135 may notify the user, via the end user device 110, of the data that was imported and included in the FHIR bundle, and the data that was unable to be imported, if any. Alternatively, and if the user only has a paper copy of the medical record, the user may, via end user device 110, utilize one or more client-facing UIs interfaces to manually enter the data via the end user device 110 to the MWT system 105. The MWT system 105 may receive the manually inputted data via the end user device 110 over the network 120, such that the data is imported to the MWT system 105. The data importer 135 may generate an FHIR bundle from the data manually provided by the user. The data importer may store the data, provided via the end user device 110, on the one or more DBs 160 with the end user identifier and/or the data source identifier utilizing ID index 200.

The data source may also be other systems or devices such a medical, wellness, training/performance systems or device. The MWT system 105 may obtain data (e.g., medical, wellness, and training/performance data) from the other system/devices in a similar manner as described above. Specifically, and if the system/device has online capability, the MWT system 105 may import the data from the other system/devices as described above, and the data importer 135 may parse the data and create a FHIR bundle. In addition or alternatively, the MWT system 105 may receive the data of the other system/device from the end user device 110 via network 120 and create the FHIR bundle, as described above. The data importer 135 may store the FHIR bundle in the one or more DBs 160 with the end user identifier and/or the data source identifier utilizing ID index 200.

The imported data 165 may be stored on the one or more DBs 160 in a machine-readable format, such as Extensible Markup Language (XML), Health Level Seven (HL7), FHIR or any other format so that the imported data 165 can be processed by the MWT system 105 to determine the MWT information (e.g., a type (e.g., medical, wellness, or training/performance) of the imported record, an affected body system, an affected body part, a severity/impact (e.g., severity of harm or quality), and an indication whether a condition associated with the imported record is currently active/in-treatment or not), as will be described in further detail below. The current release of FHIR is release 4 and entitled “Release 4 of the HL7 Fast Healthcare Interoperability Resources (FHIR®) standard”, the contents of which are hereby incorporated by reference.

For example, a medical record imported and stored on the one or more DBs may include information such as, but not limited to, title, description and notes (e.g., unstructured data), Systematized Nomenclature of Medicine-Clinical Terms (SNOMED CT) codes (e.g., structured data), Logical Observation Identifiers Names and Codes (LOINC) (e.g., structured data), International Classification of Disease (ICD) codes (e.g., structured data), activity reason (reason for the encounter or observation), allergy intolerance, care plan activity (type of activity applied), care plan activity category (type of activity: diet, encounter, observation, procedure, etc.), care plan activity outcome (what was the outcome of the activity), clinical findings (what clinical findings were done), condition code (what type of condition was treated or discovered, condition severity, condition stage, encounter reason, medication code, observation interpretation code, observation method, observation reference range code, observation value code, plan definition type (what type of care plan was being used: mental illness, cardiology, general practice (GP), etc.), service type (type of service done: oral medicine, oral surgery, nutrition, . . . ), substance code (type of substance analyzed or given), and symptom code (type of symptoms observed).

As described, the MWT system 105 may include NLP and machine learning capabilities to determine, for an imported record (e.g., medical, wellness, and/or training data), a type (e.g., medical, wellness, or training/performance) of the imported record, an affected body system, an affected body part, a severity/impact (e.g., severity of harm or quality), and an indication whether a condition associated with the imported record is currently active/in-treatment or not.

Specifically, the NLP unit 125 may analyze the imported records and extract one or more keywords from the imported records. For each imported record, the NLP unit 125 may analyze the structured data portion to derive a context for the unstructured data portion of the same imported record, e.g., based on one or more rules. For example, the structured data portion of a given imported record may include one or more codes for particular clinical findings, symptoms, diagnoses, procedures, body structures, organisms and other etiologies, substances, pharmaceuticals, devices and specimens. The NLP unit 125 may detect such codes and apply the one or more rules to derive a context for the unstructured data portion of the given imported record. Based on the derived context, the NLP unit 125 may identify one or more keywords, and may search the unstructured data portion of the given imported record for occurrences of the one or more keywords.

In an embodiment, the NLP unit 125 may analyze the unstructured data portion for the imported record to derive a context for the structured data portion of the same imported record, e.g., based on one or more rules. For example, the unstructured data portion of a given imported record may include one or more keywords that are associated with particular clinical findings, symptoms, diagnoses, procedures, body structures, organisms and other etiologies, substances, pharmaceuticals, devices and specimens. The NLP unit 125 may, based on the analysis of the unstructured data utilizing the one or more rules, identify one or more keywords in the unstructured data. The NLP unit 125 may then populate particular fields in the structured data with one or more codes associated with the keywords, wherein the codes are indicative of a particular clinical findings, symptoms, diagnoses, procedures, body structures, organisms and other etiologies, substances, pharmaceuticals, devices and/or specimens.

The NLP unit 125 may also assign one or more predetermined weights to each element in the imported records prior to or subsequent to the extracting the keywords. For example, an element in an imported record that stores a particular type of code (e.g., System Nomenclature of Medicine-Clinical Terms (SNOMED-CT) code and/or International Classification of Diseases, Tenth Revision, Clinical Modification (ICD-10-CM code)) may be assigned a higher predetermined weight, such as a weight of 10 on a scale of 1-10, while an element in the imported record that stores an activity reason (e.g., reasons for the encounter or observation) may be assigned a lower predetermined weight, such as a weight of 3 on the scale of 1-10. The predetermined weights provide a structure, i.e., ordering, to the information in the imported records such that the machine learning knows which information is more valuable than other information. In an embodiment, a medical professional, an administrator, or an authorized user of the MWT system 105 may first determine which elements in an imported record should be assigned the predetermined weights. The association between the elements and the weights (not shown) may be stored in DBs 160. The NLP unit 125 may then assign a selected weight to a selected element, utilizing the association, when the selected element is identified in the imported record.

The ML engine 145 may determine MWT information for an imported record. The MWT information may include, but is not limited to, a type (e.g., medical, wellness, or training/performance) of the imported record, an affected body system, an affected body part, a severity/impact (e.g., severity of harm or quality), and an indication whether a condition associated with the imported record is currently active/in-treatment or not. Inputs to the ML engine 145 may include the keywords obtained by the NLP unit 125, the imported records or one or more portions thereof, and weights associated with elements in the imported records. For example, the ML engine 145 may be the language understanding service (LUIS) offered by Microsoft® Azure®. The MWT information may be stored in an MWT record with additional information, such as, but not limited to, a date associated with the imported record. In addition, the MWT information from the MWT records may be indexed such that medical professionals, athletic trainers, and other individuals may utilize one or more UIs of search engines to search the stored MWT records to, for example, identify subjects with particular conditions, symptoms, severity of harm, etc.

In some embodiments, the ML engine 145 may be implemented as a supervised machine learning system. For example, training data that includes imported records, extracted keywords, predetermined weights assigned to elements in the imported records, output records, and/or the indexed data from the MWT records may be used to train the ML engine 145.

As an example for the training, let it be assumed that two medical records are used as two of a plurality of imported records of the training data set. The two medical records may be associated with a patient fracturing her right hand and are respectively dated Jan. 1, 2018 and Apr. 5, 2018. The first medical record includes, among other things, SNOMED-CT code 20511007 (i.e., structured data portion), which is the code for a fractured hand. In addition, the first medical record includes a doctor's note (i.e., unstructured data portion) indicating that the patient fractured her hand while biking. The second medical record includes, among other things, SNOMED-CT code 20511007, which is the code for a fractured hand. In addition, the second medical record includes a doctor's note indicating that the patient's fracture is a reoccurrence of a previous fracture.

The NLP unit 125 may analyze the first medical record to search for and extract keywords the first medical record. Specifically, the NLP unit 125 analyzes the SNOMED-CT code 20511007, which is the code for a fractured hand, other codes, and/or other structured data portions in the first record to derive a context for the unstructured data portion, e.g., the free text doctor note, of the first medical record. In this example, and based on or more rules, the NPL unit 125, determines that the derived context is that the first record is a medical record (e.g., not a wellness or training/performance record), and the medical record is related to a fractured bone in a patient's hand. Based on the derived context, the NLP unit 125 may search for and extracts keywords the unstructured data portion of the first medical record. In this example, the NLP unit 125 searches for and extracts keywords “fracture” and “hand”, which are related to the derived context, from the doctor's note of the first medical record.

In addition, a medical advisory board may review and analyze the first medical record and determine that a first output for the first medical record and for the training data set includes information indicating at least that the type is “medical” (e.g., medical record), the affected body system is “skeletal”, the affected body part is “hand”, the severity/impact is “1” (i.e., low on a scale of 1-4), and the status is not in treatment since the hand has healed (e.g., false).

The NLP unit 125 may also analyze the second medical record to search for and extract keywords form the second medical record. Specifically, the NLP unit 125 analyzes the SNOMED-CT code 20511007, which is the code for a fractured hand, other codes, and/or other structured data portions in the second record to derive a context for the unstructured data, e.g., the free text doctor note. In this example, the NLP unit 125, based on the one or more rules, determines that the derived context is that the second record is a medical record (e.g., not a wellness or training/performance record), and the medical record is related to a fractured bone in a patient's hand. Based on the derived context, the NLP unit 125 searches for and extracts keywords from the unstructured data portion for the second medical record. In this example, the NLP unit 125 searches for and extracts keywords “reoccurrence” (which was not identified in the first medical record), “fracture”, and “hand”, which are related to the derived context, from the doctor's note of the second medical record.

In addition, the medical advisory board may review and analyze the second medical record and determine that a second output for the second medical record and for the training data set includes information indicating at least that the type is “medical”, the affected body system is “skeletal”, the affected body part is “hand”, the severity/impact is “3” (i.e., low on a scale of 1-4), and the status is not in treatment since the hand is now healed after the reoccurrence of the broken hand. Therefore, although the first and second medical records are associated with the same type of injury after each injury has healed, the medical advisory board determines that a severity of 3 is appropriate for the second output for the training data set because the injury was a reoccurrence.

The ML engine 145 may then apply machine learning utilizing the imported records of the training data set (e.g., the first and second medical records), the results of the NLP (e.g., extracted keywords), predetermined weights assigned to each element in the imported records of the training data set, and the output records of the training data set to train the ML engine 145 to determine, for an imported record, MWT information that is stored in an MWT record. Specifically, the MWT information may include, but is not limited, to a type (e.g., medical, wellness, or training/performance) associated with an imported record, an affected body system associated with the imported record, an affected body part associated with the imported record, a severity/impact (e.g., severity of harm or quality) associated with the imported record, and an indication whether a condition associated with the imported record is currently active/in-treatment or not

In this example, the ML engine 145 determines that the first and second outputs for the first and second medical records of the training data set are the same, except that the output for the first medical record has a severity of 1 while the second output for the second medical record has a severity of 3. As such, the ML engine 145 learns (i.e., determines) that for an imported medical record for a broken bone where a keyword of “reoccurrence” is not identified and extracted, the impact field for the output may be a value of 1. Similarly, the ML engine 145 learns (i.e., determines) that for an imported record for a broken bone where a keyword of “reoccurrence” is identified and extracted, the impact field for the output may be a value of 3.

Therefore, even though the affected body system, the affected body part, impact/severity, and status information may not be explicitly included in imported records, the ML engine 145 is capable of determining this MWT information for an imported record based on the application of machine learning as described herein. As such, and subsequent to the implementation of the NLP unit 125 and application of machine learning, the ML engine 145 may determine the MWT information for an imported record and create an MWT record that stores the MWT information and other information (e.g., date associated with the imported record).

Advantageously, the MWT information and other information stored in the newly created MWT records is useful in that the MWT system 105 can utilize the MWT information and other information to generate one or more interactive computer displays that provide pictorial representations of a subject's health, wellness, and/or training/performance condition, as will be described in further detail below. In addition, and since the MWT information from the MWT records is indexed, the MWT records may be searched through a UI by, for example, medical professionals, athletic trainers, and other individuals. For example, a medical professional may utilize end user device 110 to search his/her patients MWT information stored in MWT records 170 based on severity, or some other criteria to prioritize the patients such that those patients that have higher severity of harm are seen first and/or more often.

The example as described above that uses the two medical records for a broken hand for training the ML engine 145 is for illustrative purposes only, and it is expressly contemplated that the ML engine 145 may apply more complex and different machine learning techniques with much larger sets of training data to determine the MWT information for the imported records. In addition, although the example as described above uses two medical records associated with a human being, it is expressly contemplated that the ML engine 145 may create MWT records for an animal (e.g., dog, cat, horse, etc.) based on imported records (e.g., medical, wellness, and/or training/performance data) associated with the animal and stored in databases 160 in a similar manner as described above and according to the one or more embodiments described herein. For example, the medical data for a horse may be from a medical record created by a veterinarian, whereas wellness data may be sleep, behavioral, and/or food consumption created by a caretake of with the animal. Training data may, for example, be performance data for a racehorse that is provided by the horse's trainer.

In addition or alternatively, the ML engine 145 may create MWT records for a plant and/or vegetation based on imported records (e.g., medical, wellness, and/or training/performance data) associated with the plant and/or vegetation and stored in database 160 in a similar manner as described above and according to the one or more embodiments described herein. For example, the medical data may be associated with harm to body parts the plant (e.g., harm to leaf, flower bud, stem, root, etc.), while wellness data may be associated with the amount of rain and/or sun that the plant is subjected to in a particular time period. Training data may, for example, be data indicative of the plant's reaction to particular supplements and/or chemicals (e.g., potassium, fertilizer, etc.). In a different example, the medical data may be associated with harm to an area/region of vegetation (e.g., harm to an area/region of a vineyard), while wellness data may be associated with the amount of rain and/or sun that area/region of vegetation is subjected to in a particular time period. Training data may, for example, be data indicative of the area/region of vegetation's reaction to particular supplements and/or chemicals (e.g., potassium, fertilizer, etc.).

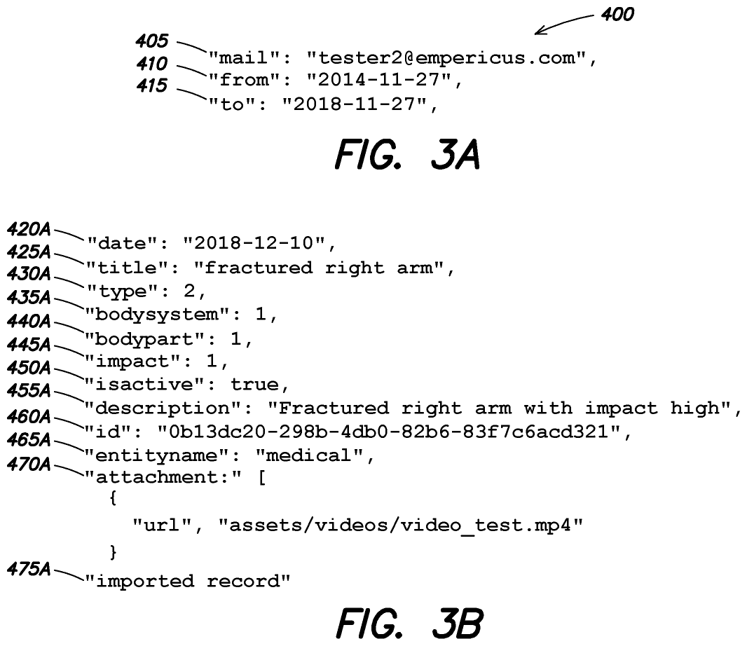

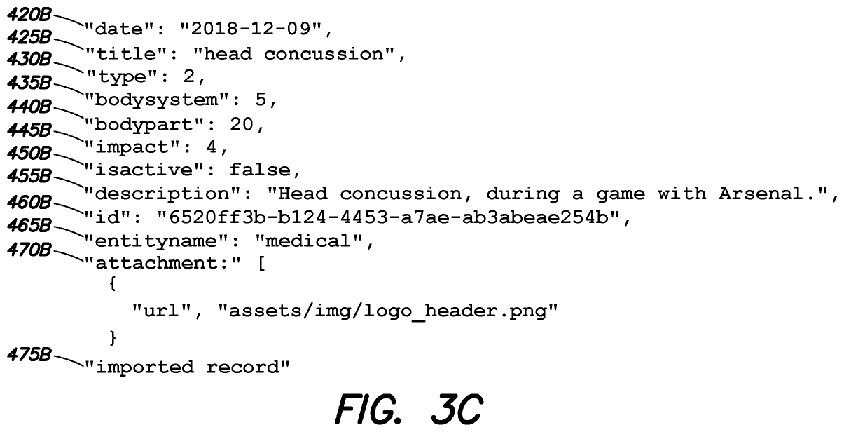

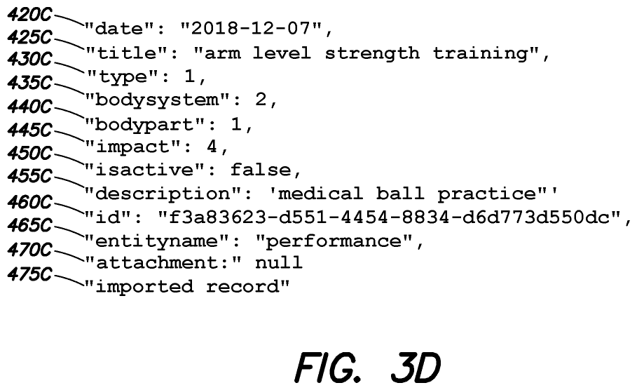

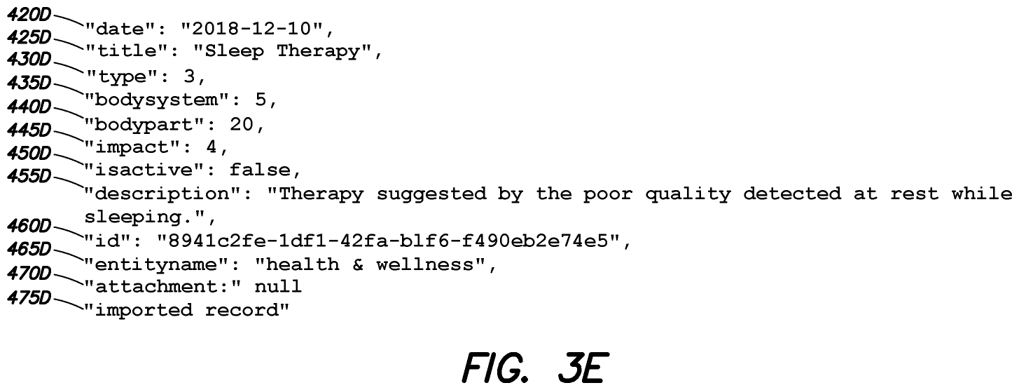

FIG. 3A is a schematic illustration of an example header 400 for one or more MWT records in accordance with one or more embodiments described herein. Specifically, header 400 includes an identifier field 405 that stores an identifier associated with a registered user of the MWT system 105. In this example, identifier field 405 stores “Tester2@MWT.com” that is the unique identifier assigned to the registered user. The ML engine 145 may obtain the identifier from the ID index 200 and store the identifier in field 405. In addition, header 400 includes a from field 410 and a to field 415 that define a time range for which the one or more MWT records, created from the registered user's (e.g., Tester2) imported records, fall within. The ML engine 145 may obtain the time range based on the MWT records stored for the registered user, and then store the time range in the from field 410 and the to field 415. The ML engine 145 may store the header 400 in the one or more DBs 160 with the imported records and/or the one or more MWT records to associate (e.g., link) the one or more MWT records and/or imported records with the registered user.

FIGS. 3B-3E are schematic illustrations of example MWT records that store the MWT information, determined based on the application of machine learning as described herein, and other information for an imported record in accordance with one or more embodiments described herein. In this example, the MWT records of FIGS. 3B-3E, created by the ML engine 145, are associated with registered user “Tester2”.

For example, the ML engine 145 may create the MWT records of FIGS. 3B-3E for four respective imported records. Specifically, and subsequent to implementing the NLP unit 125 and applying machine learning as described herein, the ML engine 145 may respectively determine the MWT information (e.g., a type, an affected body system, an affected body part, a severity/impact, and an indication whether a condition associated is currently active/in-treatment or not) for each of the four imported records. The ML engine 145 may then create an MWT record (FIGS. 3B-3E) for each imported record that stores the MWT information and other information.

Each MWT record, as depicted in FIGS. 3B-3E, include a date field (420A-420D), a title field (425A-425D), a type field (430A-430D), a bodysystem field (435A-435D), a bodypart field (440A-440D), an impact (i.e., severity) field (445A-445D), an isactive field (450A-450D), a description field (455A-455D), an id field (460A-460D), an entity name field (465A-465D), an attachment field (470A-470D), and an imported record field (475A-475D).

The date field (420A-420D) may indicate a date associated with the creation of the medical, wellness, and/or training data, e.g., imported record. For example, if a doctor creates a medical record on a particular date during a patient visit, the MWT record created for that medical record by the ML engine 145 includes a date field that stores the particular date. The ML engine 145 may extract the date from the imported record and may store the date into the date field (420A-420D). The title field (425A-425D) may indicate a title for the MWT record. For example, if a patient visits a doctor for a broken finger, the MWT record created for that medical record may include a title indicating “broken finger.” The ML engine 145 may extract a title from the imported record and store the title into the title field (425A-425D).

The type field (430A-430D) may be utilized to differentiate between, for example, medical, wellness, and training/performance MWT records that are respectfully created from imported medical, wellness, and training/performance records. As such, a first identifier (e.g., numerical value of 2) may be utilized to identify medical MWT records, a second identifier (e.g., numerical value of 3) may be utilized to identify wellness MWT records, a third identifier (e.g., numerical value of 1) may be utilized to identify training/performance MWT records, and a fourth identifier (e.g., numerical value of 0) may be utilized to indicate that the type is unknown. The ML engine 145, trained based on the application of machine learning as described herein, may determine the type to indicate whether the imported record is associated with medical, wellness, or training/performance. The ML engine 145 may store the determined type in the type field (430A-430D) of the created MWT record.

The bodysystem field (435A-435D) may indicate the affected body system associated with the imported record (e.g., medical, wellness, or training/performance) for which the MWT record is created. Specifically, each of eleven different system of the human body may be assigned a different numerical value. For example, the skeletal system may be assigned a numerical value of 1, the muscular system may be assigned a numerical value of 2, the digestive system may be assigned a numerical value of 3, the respiratory system may be assigned a numerical value of 4, the nervous system may be assigned a numerical value of 5, the circulatory system may be assigned a numerical value of 6, the endocrine system may be assigned a numerical value 7, the exocrine system may be assigned a numerical value of 8, the lymphatic system may be assigned a numerical value of 9, the renal system may be assigned a numerical value of 10, and the reproductive system may be assigned a numerical value of 11. The ML engine 145, trained based on the application of machine learning as described herein, may determine the affected body associated with the imported record. The ML engine 145 may store the determined affected body system in the bodysystem field (435A-435D) of the created MWT record.

The bodypart field (440A-440D) may indicate the affected body part associated with the imported record (e.g., medical, wellness, or training/performance) for which the MWT record is created. Specifically, each body part of the human anatomy may be assigned a different identifier (e.g., numerical value). The ML engine 145, trained based on the application of machine learning as described herein, may determine the affected body part associated with the record. The ML engine 145 may store the determined affected body part in the bodypart field (440A-440D) of the created MWT record.

The impact, i.e., severity, field (445A-445D) may indicate a severity of harm or quality associated with the imported record (e.g., medical, wellness, or training/performance) for which the MWT record is created. Specifically, and for medical MWT records (e.g., the type field stores a value indicating a medical MWT record) created for medical records, an impact scale may be utilized to indicate severity of harm. For example, the impact scale may be from 1 to 4. A value of 1 may indicate minimal severity of harm (e.g., healthy), a value of 2 may indicate mild severity of harm, a value of 3 may indicate moderate severity of harm, and a value of 4 may indicate severe severity of harm. The ML engine 145, trained based on the application of machine learning as described herein, may determine the impact/severity of harm associated with the imported medical record. The ML engine 145 may store the determined impact/severity of harm in the impact, i.e., severity, field (445A-445D) of the created medical MWT record.

For wellness and training/performance records (e.g., the type field stores a value indicating a wellness MWT record or a training/performance record), an impact scale may be utilized to indicate severity of quality. For example, the impact scale may be from 1 to 4. A value of 1 may indicate minimal wellness quality, a value of 2 may indicate low/mild wellness quality, a value of 3 may indicate moderate wellness quality, and a value of 4 may indicate severe/high wellness quality. Similarly, a value of 1 may indicate minimal training/performance quality, a value of 2 may indicate low/mild training/performance quality, a value of 3 may indicate moderate training/performance quality, and a value of 4 may indicate severe/high training/performance quality. The ML engine 145, trained based on the application of machine learning as described herein, may determine an impact/severity of quality associated with the imported wellness and/or training/performance record. The ML engine 145 may store the determined impact/severity of quality in the impact, i.e., severity, field (445A-445D) of the created wellness and/or training performance MWT records.

The isactive field (450A-450D) indicates if a condition indicated in the imported record, for which the MWT record is created, is currently active/in-treatment or not. Specifically, the isactive field may include either one of two values. The first value (e.g., true or a numerical value of 1) may indicate that the condition is active/in treatment. The second value (e.g., false or a numerical value of 0) may indicate that the condition is not active/in-treatment. The ML engine 145, trained based on the application of machine learning as described herein, may determine the active/in-treatment or not status associated with the imported medical record. The ML engine 145 may update the isactive field (450A-450D) at a time when a condition is determined to have finished, treatment has transitioned from active to non-active, or transitions from non-active to active. The ML engine 145 may store the derived active/not-active status in the isactive field (450A-450D) of the created MWT record.

The description field (455A-455D) may store a description of the MWT record. For example, if the MWT record is created from a medical record associated with a patient's broken thumb, the description field may indicate “fractured thumb” or a similar phrase. The ML engine 145 may, for example, extract the description from the imported record and store the description in the description field (455A-455D) of the MWT record. The id field (460A-460D) may store a unique identifier to distinguish each MWT record from one another. The ML engine 145 may, for example, generate and/or assign a unique identifier to each created MWT record and store the unique identifier in the id field (460A-460D) of the created MWT record. The entityname field (465A-465D) may store, in text form, the type of MWT record or other relevant information for the MWT record. For example, the entityname field may be based on the type field (430A-430D). The ML engine 145 may, for example, extract an entity name from the imported record and store the entity name in the entityname field (465A-465D) of the created MWT record.

The attachment field (470A-470D) may store one or more links to one or more files or data objects associated with the imported record for which MWT record is created. For example, the attachment field may store a link to an x-ray scan, a computer tomography (CT) scan, video, picture, etc. The ML engine 145 may, for example, extract an attached file or data object from the imported record and store a link to the attachment in the attachment field (470A-470D) of the MWT record. If no attachments exist for the record, the attachment field may store a null value.

In addition, the created MWT records may be linked to the original imported record. The ML engine 105 may store one or more links (e.g., a pointer) to the imported record, for which the MWT record is created, in imported record field (475A-475D). The ML engine 145 may, for example, determine a storage location of the imported record and store the storage location in the imported record field (475A-475D) of the created MWT record such that the imported record and the created MWT record are linked/associated with each other.

FIGS. 3B and 3C are schematic illustration of medical MWT records created by the ML engine 145 for imported medical records. The MWT record of FIG. 3B is associated with an imported medical record for Tester2 created on Dec. 10, 2018 for the fracture of the right arm. As such, the bodysy stem field 435A stores a value of 1 indicating that the skeletal system was the affected body system, which may be determined by the ML engine 145 trained based the application of machine learning as described herein. In addition, the bodypart field 440A stores a value of 1 to indicate that the right arm was the affected body part, which may be determined by the ML engine 145 trained based on the application of machine learning as described herein. Further, the impact field 445A stores a value of 1 indicating minimal severity of harm/healthy, which may be determined by the ML engine 145 trained based on the application of machine learning as described herein. Moreover, the isactive field 450A stores a value of “true” indicating that the fracture right arm is still fractured or in treatment (e.g., physical therapy), which may be determined by the ML engine 145 trained based on the application of machine learning as described herein. The ML engine 145 may store a link, e.g., URL, for a video of how the fracture occurred in the attachment field 470A. In addition, a link or association to the imported medical record, for which the medical MWT record of FIG. 3B is created, may be stored in field. 475A.

The MWT record of FIG. 3C is associated with an imported medical record for Tester2 created on Dec. 9, 2018 for a head concussion. As such, the bodysystem field 435B stores a value of 5 indicating that the nervous system was the affected body system, which may be determined by the ML engine 145 trained based on the application of machine learning as described herein. In addition, the bodypart field 440B stores a value of 20 indicating that the head is affected body part, which may be determined by the ML engine 145 trained based on the application of machine learning as described herein. Further, the impact field 445B stores a value of 2 indicating that the severity of harm is mild, which may be determined by the ML engine 145 trained based on the application of machine learning as described herein. Further, the isactive field 450B stores a value of “false” indicating that the head concussion is no longer active and there is no current treatment for the suffered head concussion, which may be determined by the ML engine 145 trained based on the application of machine learning as described herein. The ML engine 145 may store a link to an image of Tester2's brain scan in the attachment field 470B. In addition, a link or association to the imported medical record, for which the medical MWT record of FIG. 3C is created, may be stored in field. 475B.

FIG. 3D is an illustrative training/performance MWT record created by the ML engine 145 for an imported training/performance record in accordance with one or more embodiments described herein. The MWT record of FIG. 3D is associated with a training/performance record for Tester2 created on Dec. 7, 2018 for arm strength training. As such, the bodysystem field 435C stores a value of 2 to indicate that the muscular system was the affected body system, which may be determined by the ML engine 145 trained based on the application of machine learning as described herein. In addition, the bodypart field 440C may store a value of 1 to indicate that the right arm was the affected body part, which may be determined by the ML engine 145 trained based on the application of machine learning as described herein. Further, the impact field 445C stores a value of 4 indicating that the quality of the training/performance is severe/high (e.g., rigorous training session to strengthen right arm), which may be determined by the ML engine 145 trained based on the application of machine learning as described herein. Moreover, the isactive field 450C stores a value of “false” indicating that the arm strength training is no longer active, which may be determined by the ML engine 145 trained based on the application of machine learning as described herein. There is no attachment for this training/performance record, and as such the ML engine 145 may store a null value in the attachment field 470C. In addition, a link or association to the imported training/performance record, for which the training/performance MWT record of FIG. 3D is created, may be stored in field. 475C.

FIG. 3E is an illustrative wellness MWT record created by the ML engine 145 for an imported wellness record in accordance with one or more embodiments described herein. The MWT record of FIG. 3E is associated with a wellness record for Tester2 created on Dec. 12, 2018 for sleep therapy. As such, the bodysystem field 435D stores a value of 5 to indicate that the nervous system was the affected body system, which may be determined by the ML engine 145 trained based on the application of machine learning as described herein. In addition, the bodypart field 440D stores a value of 20 to indicate that the head was the affected body part, which may be determined by the ML engine 145 trained based on the application of machine learning as described herein. Further, the impact field stores a value of 4 indicating that the wellness quality is severe/high (e.g., sleep therapy has a high impact on good sleep quality), which may be determined by the ML engine 145 trained based on the application of machine learning as described herein. Moreover, the isactive field 450D stores a value of “false” indicating that sleep therapy is no longer active, which may be determined by the ML engine 145 trained based on the application of machine learning as described herein. There is no attachment for this wellness record, and as such the ML engine 145 may store a null value in the attachment field 470D. In addition, a link or association to the imported wellness record, for which the wellness MWT record of FIG. 3E is created, may be stored in field. 475D.

The structure of the header and the MWT records in FIGS. 3A-3E and the values and information stored therein are for illustrative purposes only. It is expressly contemplated that the MWT records described herein may have different structures and may include less, additional, or other information. For example, although the MWT records of FIGS. 3B-3E store a single value in the type field, the bodysystem field, and the bodypart field, it is expressly contemplated that a plurality of values may be stored in each field. In addition, although reference is made to the records of FIGS. 3A-3E being created for a human being, it is expressly contemplated that the MWT records as described herein may be created for different type of subjects. For example, the ML engine 145 may create one or more MWT records 300 for an animal (e.g., dog, cat, horse, etc.) in a similar manner as described above and utilizing imported records associated with the animal that is stored in databases 160 according to the one or more embodiments described herein. In addition or alternatively, the ML engine 145 may create one or more MWT records for a plant and/or vegetation in a similar manner as described above and utilizing imported records associated with the plant and/or vegetation that is stored in database 160 according to the one or more embodiments described herein.

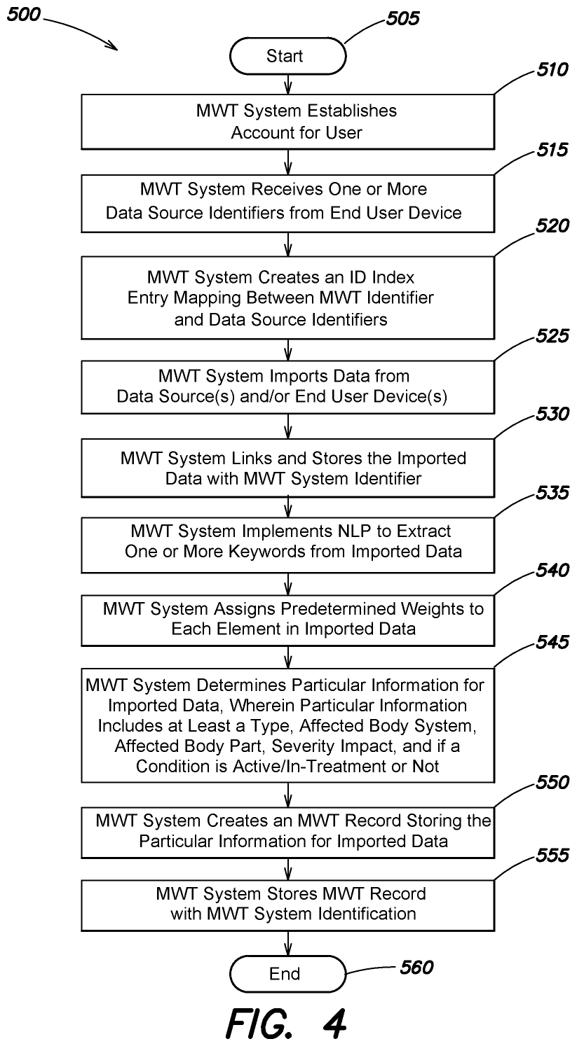

FIG. 4 is a schematic illustration of s flow diagram of an example method for creating an MWT record in accordance with one or more embodiments described herein. The procedure 500 starts at step 505 and continues to step 510 where the MWT system 105 establishes an account for a user.

For example, a user may utilize an end user device 110 to access one or more UIs, e.g., webpages, associated with the MWT system 105, and then utilize the client-facing UIs interfaces to provide personal information (e.g., name, date of birth, address, social security number, etc.). The MWT system 105 may utilize the personal information to establish a unique MWT system account for the user (e.g., register the user with the MWT system 105). Subsequently, the user may provide user credentials (e.g., username and password) to the MWT system 105, and the MWT system 105 may provide the user with access to the user's unique MWT system account.

The procedure continues to step 515 where the MWT system 105 receives one or more data source identifiers from the end user device. Specifically, the MWT system 105 may receive the data source identifiers over the network 120 and from the end user device 110, e.g., inputted by a user. The procedure continues to step 520 where the MWT system creates an ID index entry mapping the user's MWT system identifier to one or more data source identifiers. Specifically, the MWT system 105 may store the user's MWT system identifier in the first section 205 and store the user's one or more data source identifiers in second section 210 of the ID index 200.

The procedure continues to step 525 where the MWT system 105 imports data (e.g., medical, wellness, and/or training/performance data), associated with a subject, from one or more data sources and/or one or more end user devices. Specifically, the data importer 135 may import medical, wellness, and/or training/performance data (e.g., records) from EMRS, electronic medical devices, health devices, wellness devices, training devices, and/or performance devices. In addition or alternatively, the MWT system 105 may import medical, wellness, and/or training data from the end user device 110. The data importer 135 may then store the medical, wellness, and/or training/performance data as, for example, an FHIR bundle, on the one or more DBs 160 with the user's MWT system identifier.

It is noted that the imported medical, wellness, and/or training/performance data (e.g., records) may be data associated with the user that established the unique MWT system account utilizing his/her information. Alternatively, the imported medical, wellness, and/or training/performance data (e.g., records) may be data associated with a different subject (e.g., a different user, animal, plant, vegetation, etc.) than the user who establishes the unique MWT system account. For example, an adult may establish the unique MWT system account utilizing his/her personal information, and the importer 135 may import medical, wellness, and/or training/performance data (e.g., records) for the parent's child/infant. In addition or alternatively, the imported medical, wellness, and/or training performance data (e.g., records) may be data associated with an animal, plant, vegetation, and/or a different type of subject owned or under the control of the user that established the unique MWT system account.

The procedure continues to step 530 and the MWT system 105 links and stores the imported data with the MWT user identifier associated with the user. Specifically, the MWT system 105 may, for example, extract a data source identifier for the imported record. The MWT system 105 may then determine if the extracted data source identifier is stored in second section 210 of the ID index 200. If so, the MWT system 105 may obtain the corresponding MWT system identifier that is stored in the corresponding first section 205 of the ID index 200. The MWT system 105 may then store the imported record on the one or more DBs 160 with the MWT system identifier associate with the user to link the data with the user.

The procedure continues to step 535 and the MWT system 105 implements NLP to extract one or more keywords from the imported data. Specifically, the NLP unit 125 may extract the one or more keywords from the imported records. For each imported record, the NLP unit 125 may analyze the structured data portion to derive a context for the unstructured data portion of the same imported record, e.g., based on one or more rules. For example, the structured data portion of a given imported record may include one or more codes for particular clinical findings, symptoms, diagnoses, procedures, body structures, organisms and other etiologies, substances, pharmaceuticals, devices and specimens. The NLP unit 125 may detect such codes and apply the one or more rules to derive a context for the unstructured data portion of the given imported record. Based on the derived context, the NLP unit 125 may identify one or more keywords, and may search the unstructured data portion of the given imported record for occurrences of the one or more keywords.

In addition or alternatively, the NLP unit 125 may analyze, for each imported record, the unstructured data portion to derive a context for the structured data portion of the same imported record, e.g., based on one or more rules. For example, the unstructured data portion of a given imported record may include one or more keywords that are associated with particular clinical findings, symptoms, diagnoses, procedures, body structures, organisms and other etiologies, substances, pharmaceuticals, devices and specimens. The NLP unit 125 may, based on the analysis of the unstructured data utilizing the one or more rules, identify one or more keywords in the unstructured data. The NLP unit 125 may then populate particular fields in the structured data with one or more codes associated with the keywords, wherein the codes are indicative of a clinical findings, symptoms, diagnoses, procedures, body structures, organisms and other etiologies, substances, pharmaceuticals, devices and/or specimens.

The procedure continues to step 540 and the MWT system 105 assigns predetermined weight to each element in the imported data. Specifically, the NLP unit 125 may assign predetermined weight to each element in the imported record prior to or subsequent to the extracting the keywords. For example, an element in a imported record may store a particular type of code (SNOMED-CT and/or ICD code) that may be assigned a higher predetermined weight, such as 10 on a scale of 1-10, while an element in the imported record storing an activity reason (e.g., reasons for the encounter or observation) may be assigned a lower predetermined weight, such as 3 on the scale of 1-10. The predetermined weights provide a structure to the information in the imported records such that the machine learning knows which information is more important than other information. In an embodiment, the predetermined weights, assigned to each element in an important record, may be first determined by a medical professional, an administrator, or an authorized user of the MWT system 105.

The procedure continues to 545 and the MWT system 105 determines MWT information for the imported record, wherein the MWT information includes, but is not limited to, a type (e.g., medical, wellness, or training/performance) of the imported record, an affected body system, an affected body part, a severity/impact (e.g., severity of harm or quality), and an indication whether a condition associated with the imported record is currently active/in-treatment or not. Specifically, the ML engine 145 may determine MWT information for each imported record associated with the user. Inputs to the ML engine 145 to train the ML engine 145 may include the keywords obtained by the NLP unit 125, the imported records or one or more portions thereof, and weights associated to elements in the imported records.

The procedure continues to step 550 and the MWT system 105 creates an MWT record for the imported data. Specifically, the ML engine 145, trained based on the application of machine learning as described herein, may create a different MWT record for each imported record and store the MWT information for the imported record in an MWT record. The MWT record may also store additional information that includes, but is not limited to, a date associated with the imported record. The procedure continues to step 555 and the MWT system 105 stores the MWT record with the MWT system identifier associated with the user on the on the one or more DBs 160. The procedure ends at step 560.

The MWT records, created by the ML engine 145 subsequent to the implementing NLP and the application of machine learning, may be utilized by the MWT system 105 to generate one or more interactive computer displays that provide pictorial representations of a subject's health, wellness, and/or training/performance, as described herein.

Specifically, lifeline generator 130 may generate a different interactive computer-generated lifeline for each type of data (e.g., medical, wellness, or training/performance) indicated in the MWT records. Each interactive computer-generated lifeline may be a longitudinal, vertical, diagonal, or any shaped graph that is segmented according to a particular time interval (e.g., days, months, or years). The MWT system 105 may, for each time interval on the interactive computer-generated lifeline, aggregate together the one or more MWT records that indicate a date that falls within the same time interval. The MWT system 105 may, for each time interval, include or omit an element (e.g., marking) from the interactive computer-generated lifeline and determine a size of the element based on, for example, the severity/impact and/or or other information indicated in the one or more MWT records that make up the aggregate for the time interval.

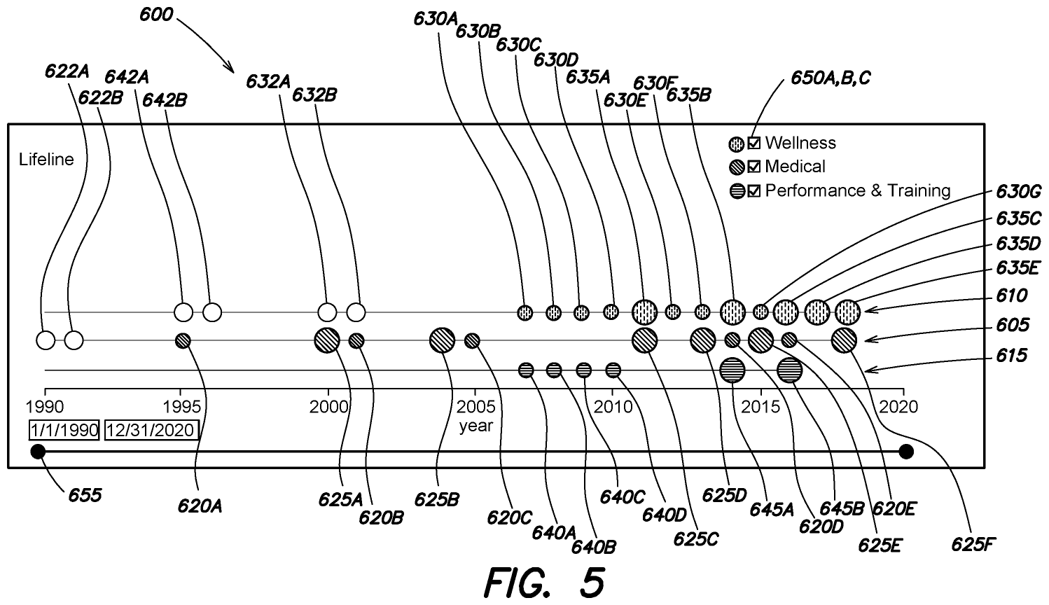

FIG. 5 is a schematic illustration of an example computer display depicting interactive computer-generated lifelines in accordance with one or more embodiments. Specifically, a user may provide his/her user credentials via an end user device 110 to gain access to the user's account. The MWT system 105 may then display, on an end user device 110, the illustrative interactive computer-generated lifelines that are generated based on the MWT record created for a particular subject's (e.g., human being, animal, plant, vegetation, etc.) imported records.

The computer display 600 includes a medical interactive computer-generated lifeline 605, a wellness interactive computer-generated lifeline 610, and a training/performance interactive computer-generated lifeline 615. The lifeline generator 130 may visually differentiate each of the lifelines from each outer utilizing shading, coloring, or any of a variety of different graphical affordances. In addition, the lifeline generator 130 may segment/organize each interactive computer-generated lifeline according to a particular time interval (e.g., day, month, year, etc.). The illustrative interactive computer-generated lifelines, e.g., 605, 610, and 615, of computer display 600, are segmented and organized by the lifeline generator 130 according to a yearly time interval.

To generate the medical interactive computer-generated lifeline 605, the lifeline generator 130 may analyze the stored MWT records for a particular subject and create a different aggregate (e.g., a grouping) for each set of medical MWT records (e.g., type field stores a value of 2) that indicate a date that falls within the same time interval. For example, the lifeline generator 130 may aggregate all medical MWT records for a particular subject (e.g., based on the header with the same MWT system identifier) that fall within the year of 1990 to form a first aggregate of medical MWT records. In addition, the lifeline generator 130 may aggregate all medical MWT records for the particular subject that fall within the year of 1991 to form a second aggregate of medical MWT records, and so forth.

The lifeline generator 130 may then determine, for each time interval, whether a marking should be included at the time interval on the medical interactive computer-generated lifeline 605 and the size of the marking based on the severity/impact indicated in the set of medical MWT records that make up each aggregate for the time interval. For example, each marking may be one of a plurality of sizes, e.g., a small sized circle, a medium sized circle, or a large sized circle. No marking may represent minimal severity of harm/healthy, a small sized marking may represent mild severity of harm, a medium sized mark may represent a moderate severity of harm, and a large sized marking may represent a severe/high severity of harm.

For example, the lifeline generator 130 may implement an algorithm that indicates that no marking (i.e., omit a marking), indicating minimal severity of harm/healthy, should be included at the time interval on the medical interactive computer-generated lifeline 605 to represent the aggregate if all medical MWT records in the aggregate for the time interval indicates an impact/severity value of 1. The algorithm may also indicate that a small sized marking, indicating mild severity of harm, should be included at the time interval on the medical interactive computer-generated lifeline 605 to represent the aggregate if any medical MWT record in the aggregate for the time interval indicates an impact/severity of at least 2 and not greater than 2. Further, the algorithm may indicate that a medium size marking, indicating moderate severity of harm, should be included at the time interval on the medical interactive computer-generated lifeline 605 to represent the aggregate if any medical MWT record in the aggregate for the time interval indicates an impact/severity of at least 3 and not greater than 3. In addition, the algorithm may indicate that a large size marking, indicating severe severity of harm, should be included at the time interval on the medical interactive computer-generated lifeline 605 to represent the aggregate if any medical MWT record in the aggregate indicates an impact/severity of at least 4.

Let it be assumed that the lifeline generator 130 utilized this algorithm to generate the medical interactive computer-generated lifeline 605 of computer display 600. Therefore, the small sized markings 620A-620E indicate mild severity of harm for years 1995, 2001, 2005, 2014, and 2016, and that each aggregate corresponding to years 1995, 2001, 2005, 2014, and 2016 includes at least one medical MWT record that indicates an impact/severity of at least 2 and not greater than 2. The medium sized markings 622A and 622B indicate moderate severity of harm for years 1990 and 1991, and that each aggregate corresponding to years 1990 and 1991 includes at least one medical MWT record that indicates an impact/severity of at least 3 and not greater than 3. The large sized markings 625A-625F indicate severe severity of harm for years 2000, 2004, 2011, 2013, 2015, and 2018, and that each aggregate corresponding to years 2000, 2004, 2011, 2013, 2015, and 2018 includes at least one medical MWT record that has an impact field value of 4. No markings indicate minimal severity of harm/healthy at years 1992, 1993, 1994, 1996, 1997, 1998, 1999, 2002, 2003, 2006, 2007, 2008, 2009, 2010, 2012, 2017, 2019, and 2020, and that each aggregate corresponding to years 1992, 1993, 1994, 1996, 1997, 1998, 1999, 2002, 2003, 2006, 2007, 2008, 2009, 2010, 2012, 2017, 2019, and 2020 includes medical MWT records that indicate an impact/severity that is not greater than 1.

The algorithm described above is for illustrative purposes only, and it is expressly contemplated that the lifeline generator 130 may utilize any of a variety of different algorithms in conjunction with different and/or addition information indicated in medical MWT records to determine if a marking should be included on the medical interactive computer-generated lifeline 605 and the size of the marking on the medical interactive computer-generated lifeline 605. For example, the lifeline generator 130 may utilize the impact field in conjunction with the isactive field, and/or number of occurrences (e.g., if a plurality of medical MWT records include a date within a particular time period) to determine if a marking should be included and the size of the marking.

As such, the medical interactive computer-generated lifeline 605 is a visual tool that provides a pictorial representation, such as a linear representation, of a subject's medical history over time, where the areas with the highest level of interest, e.g., severity/impact of harm, are emphasized, e.g., highlighted by the markings.

The lifeline generator 130 may generate the wellness interactive computer-generated lifeline 610 and the training/performance interactive computer-generate lifeline 615 in a similar manner.

To generate the wellness interactive computer-generated lifeline 610, the lifeline generator 130 may analyze the stored MWT records for the particular subject and create a different aggregate for each set of wellness MWT records (e.g., type field stores a value of 3) that indicate a date that falls within the same time interval. For example, the lifeline generator 130 may aggregate together all wellness MWT records for the particular subject that fall within the year of 1990 to form a first aggregate of wellness MWT records. In addition, the lifeline generator 130 may aggregate together all wellness MWT records for the particular subject that fall within the year of 1991 to form a second aggregate of wellness MWT records, and so forth.

The lifeline generator 130 may determine, for each time interval, whether a marking should be included at the time interval on the wellness interactive computer-generated lifeline 610 and the size of the marking based on the severity/impact indicated in the set of wellness MWT records that make up each aggregate for the time interval. For example, no marking may indicate minimal wellness quality, e.g., wellness MWT record for subject's sleep behavior indicates that subject has insomnia. A small sized marking 630A-630G may represent low/mild wellness quality, e.g., wellness MWT record for subject's sleep behavior indicates that the subject wakes up throughout the night. In addition, a medium sized marking 632A and 632B indicates moderate wellness quality e.g., wellness MWT record for subject's sleep behavior indicates that the subject sleeps while only waking up a few times, and a large sized marking 635A-635E may represent severe/high wellness quality, e.g., wellness MWT record for subject's sleep behavior indicates that the subject sleeps throughout the night. As described above with reference to the generation of the medical interactive computer-generated lifeline 605, the lifeline generator 130 may analyze the impact/severity indicated and/or other information, and utilize any of a variety of different algorithms to determine if a marking should be included at the time interval on the wellness interactive computer-generated lifeline 610 and the size of the marking on the wellness interactive computer-generated lifeline 610. For example, the lifeline generator 130 may utilize the impact field in conjunction with number of occurrences (e.g., if a plurality of wellness MWT records include a date within a particular time period) to determine if a marking should be included and the size of the marking.

As such, the wellness interactive computer-generate lifeline 610 is a visual tool that provides a pictorial representation, such as a linear representation, of a subject's wellness history over time, where the areas with the highest level of interest, e.g., wellness quality, are highlighted by the markings.

To generate the training/performance interactive computer-generated lifeline 615, the lifeline generator 130 may analyze the stored MWT records for the particular subject and create a different aggregate for each set of training/performance MWT records (e.g., type field stores a value of 1) that indicate a date that falls within the same timer interval. For example, the lifeline generator 130 may aggregate together all training/performance MWT records for the particular subject that fall within the year of 1990 to form a first aggregate of training/performance MWT records. In addition, the lifeline generator 130 may aggregate together all training/performance MWT records for the particular subject that fall within the year of 1991 to form a second aggregate of training/performance MWT records, and so forth.

The lifeline generator 130 may determine, for each time interval, whether a marking should be included at the time interval on the training/performance interactive computer-generated lifeline 615 and the size of the marking based on the severity/impact indicated in the set of training/performance MWT records that make up each aggregate for the time interval. For example, no marking may indicate minimal training/performance quality, e.g., no associated training/performance data or the training/performance MWT record indicates that the subject is not working out or training. A small sized marking 640A-640D may indicate low/mild training/performance quality, e.g., training/performance MWT record indicates that the subject is working out sporadically with minimal effort/exertion. A medium sized marking 642A and 642B may indicate moderate training/performance quality, e.g., training/performance MWT record indicates that the subject is working out consistently, and a large sized marking 645A and 645B may indicate severe/high training/performance quality, training/performance MWT record indicates that the subject is working consistently and rigorously. As described above with reference to the generation of the medical interactive computer-generated lifeline 605, the lifeline generator 130 may analyze the impact/severity indicated and/or other information, and utilize any of a variety of different algorithms to determine if a marking should be included at the time interval on the wellness interactive computer-generated lifeline 610 and the size of the marking on the wellness interactive computer-generated lifeline 610. For example, the lifeline generator 130 may utilize the impact field in conjunction with number of occurrences (e.g., if a plurality of training/performance MWT records include a date within a particular time period) to determine if a marking should be included and the size of the marking.

As such, the training/performance interactive computer-generated lifeline 615 is a visual tool that provides a pictorial representation, such as a linear representation, of a subject's training/performance history over time, where the areas with the highest level of interest, e.g., training/performance quality, are highlighted by the markings.

In the examples provided, the impact scale utilized for the medical MWT records is from minimal severity of harm (1) to high severity of harm (4), while the impact scale utilized for the wellness and training/performance MWT records is from minimal quality (1) to high quality (2). As such, the markings on the medical interactive computer-generated lifeline 605 provide an opposite connotation than the markings on the wellness interactive computer-generated lifelines 610 and the training/performance interactive computer-generated lifelines 615. Specifically, the inclusion of the markings and the increase in size of the markings at a time intervals provide a negative connotation (e.g., harm) to a user viewing the medical interactive computer-generated lifeline 605, while the inclusion of the markings and the increase in size of the markings at the time intervals provide a positive connotation (e.g., quality) to a user viewing the wellness interactive computer-generated lifeline 610 and the training/performance interactive computer-generated lifeline 615.

The structure and appearance of the interactive computer-generated lifelines 605, 610, and 615 of computer display 600 are for illustrative purposes only, and it is expressly contemplated that the interactive computer-generated lifelines 605, 610, and 615 may be visually depicted in different ways. For example, although the interactive computer-generated lifelines 605, 610, and 615 utilize circular markings of three sizes, it is expressly contemplated that any type of markings (e.g., square, line, star, etc.) and any number of sizes may be utilized to visually indicate impact on the interactive computer-generated lifelines 605, 610, and 615 according to any type of implemented algorithm.

The computer display 600 may include toggles 650A-650C, each of which is associated with a different interactive computer-generated lifeline. As such, the lifeline generator 130 may include or exclude a corresponding interactive computer-generated lifeline from the computer display based a selection/deselection of toggles 650A-650C, e.g., by a user. In addition, the computer display may include a slider 655. The slider 655 may be manipulated by a user such that the lifeline generator 130 focuses the interactive computer-generated lifelines on one or more times of interest. For example, if the user moves the left end of the slider 655 to the year 2000 and the right end of the slider to 2010, the lifeline generator 130 will alter the display 600 such that the interactive computer-generated lifelines are displayed to only include years 2000 to 2010.

The lifeline generator 103 may, in response to the selection of a marking on the interactive computer-generated life, create and present (i.e., display) a listing of the MWT records that make up the aggregate represented by the marking at the time interval.

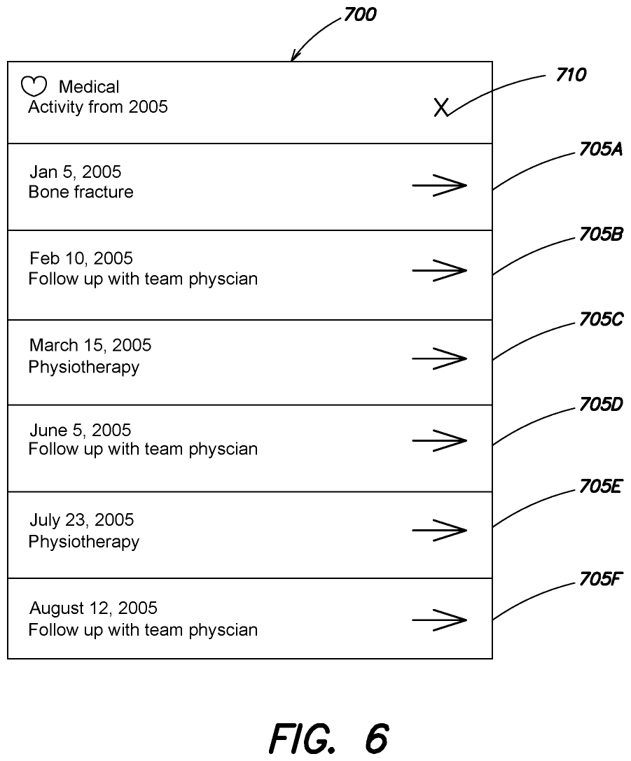

FIG. 6 is a schematic illustration of an example listing associated with the MWT records of an aggregate represented by a marking on an interactive computer-generated lifeline in accordance with one or more embodiments. For example, and with reference to FIG. 5, in response to receiving a selection, e.g., by a user, of the marking at year 2005 of the medical interactive computer-generated lifeline 605, the lifeline generator 130 may display listing 700 that is associated with the medical MWT records that make up the aggregate represented by the selected marking at year 2005. The listing includes a plurality of entries 705A-705F, each of which may correspond to a different medical MWT record of the aggregate represented by the selected marking at year 2005. In addition, the lifeline generator 130 may list the entries 705A-705F, corresponding to the MWT records, according to severity, with the entry having the highest severity being first. Alternatively, the lifeline generator 130 may utilize any other of a variety of factors to list the entries 705A-705F in any particular order. Each entry may include a date(s) and a brief description describing the entry. The lifeline generator 130 may extract the date and brief description from the associated MWT record or the corresponding imported record and store the date and brief description in the listing 700.

In response to a selection of the “X” 710, e.g., user selection, the lifeline generator 130 may close the listing 700 and revert back to the interactive computer-generated lifelines 605, 610, 615. Alternatively, and in response to a selection of a particular entry of the plurality of entries 705A-705F in the listing, the lifeline generator 103 may present details associated with the particular entry.

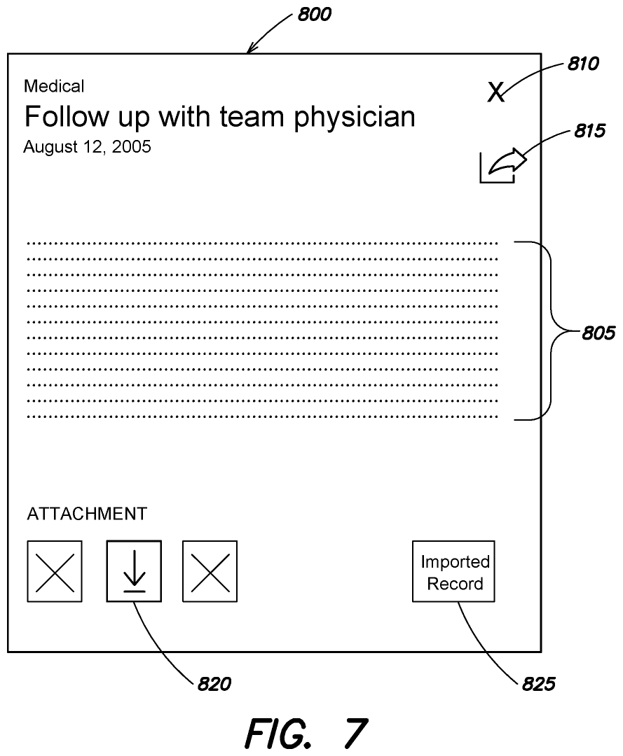

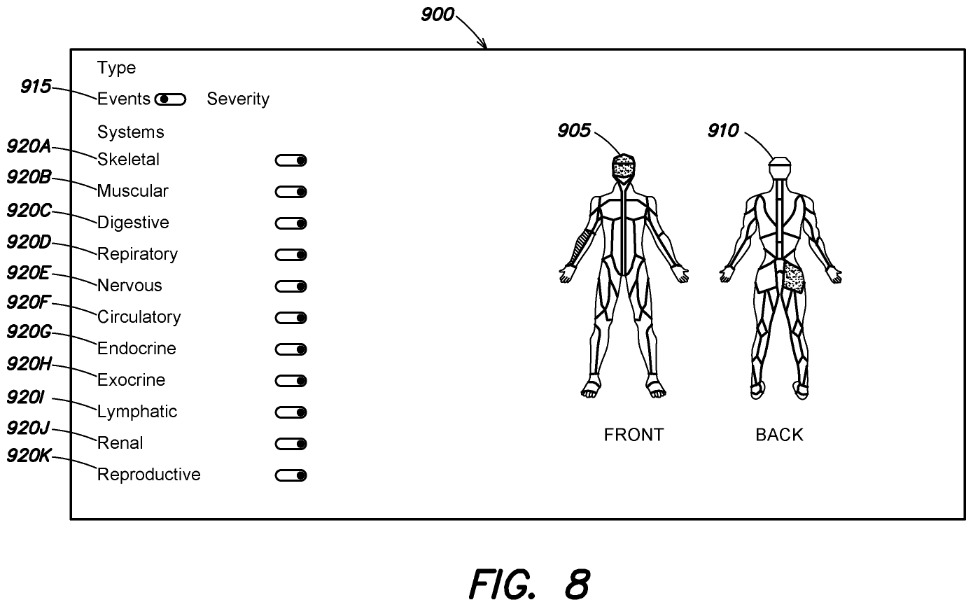

FIG. 7 is a schematic illustration of an example entry from a listing in accordance with one or more embodiments described herein. The entry 800 corresponds to a selected MWT record and includes the brief description, the date, and a detailed description field 805. The detailed description field 805 may provide the details for the entry corresponding to the selected MWT record. For this example, the detailed description 805 may include the diagnosis by the team physician during a follow-up visit. The lifeline generator 130 may extract the detailed description from the associated MWT record or the corresponding imported record and store the detailed description in detailed description field 805. In response to a selection of “X” 810, e.g., user selection, the lifeline generator 130 may revert back to the listing 700. In response to a selection of button 815, the lifeline generator 130 may provide a mechanism to forward the entry 800 (e.g., MWT record) to a different user (e.g., doctor, specialist, etc.). Further, and in response to selection of attachment button 820, the lifeline generator 130 may obtain and present, to a user, the attachment associated with the MWT record. In response to a selection of imported record button 825, the lifeline generator may obtain, from the one or more DBs 160, the linked imported record and present, to a user, the imported record for which the MWT record is created.

Therefore, a user may interact with the medical interactive computer-generated lifeline 605 by selecting one or more markings that, for example, indicate high severity of harm (i.e., negative medical history), such that the MWT system 105 provides the user with the medical details of the aggregate represented by the marking. Advantageously, the user can investigate in real-time a subject's negative medical history and the cause and attributes associated with the negative medical history. By obtaining a better understanding of the subject's medical history utilizing the medical interactive computer-generated lifeline 605, better insight can be gained for the subject's future medical prevention.

Similarly, a user may interact with the wellness interactive computer-generated lifeline 610 by selecting one or more markings that, for example, indicate wellness quality, such that the MWT system 105 provides a user with the wellness details of the aggregate represented by the marking. Advantageously, the user can investigate in real-time the subject's positive wellness history and the cause and attributes associated with the positive wellness history. Therefore, the subject may adapt his/her future wellness behavior by obtaining a better understanding of the subject's wellness history utilizing the wellness interactive computer-generated lifeline 610.

In addition, a user may interact with the training/performance interactive computer-generated lifeline 615 by selecting one or more markings that, for example, indicate training/performance quality, such that the MWT system 105 provides a user the training/performance details of the aggregate represented by the marking. Advantageously, the user can investigate in real-time a subject's positive training/performance history and the cause and attributes associated with the positive training/performance history. Therefore, the subject may adapt his/her future training/performance behavior by obtaining a better understanding of the subject's training/performance history utilizing the training/performance interactive computer-generated lifeline 615.

The body map generator 140 may also generate an interactive computer-generated body map depicting body parts of the subject's anatomy that are highlighted utilizing information from the MWT records. Specifically, a user may provide his/her user credentials via an end user device 110 to gain access to the user's account. The MWT system 105 may then display, on an end user device 110, the illustrative interactive computer-generated body map that are generated based on the MWT record created from a particular subject's imported records.

FIG. 8 is a schematic illustration of an example computer display depicting an interactive computer-generated body map that highlights body parts of a human being based on data type in accordance with one or more embodiment described herein. The interactive computer-generated body map, on computer display 900, includes a front view of a human avatar 905 and a back view of a human avatar 910. The front view of the human avatar 905 and the back view of the human avatar 910 depict body parts associated with the human anatomy. Particular body parts on the front view of the human avatar 905 and the back view of the human avatar 910 may be highlighted utilizing shading, coloring, or any of a variety of different graphical affordances.

When the toggle 915 is set to events, the body map generator 140 highlights body parts (e.g., shading, color, etc.) on the interactive computer-generated body map according to the type of data, where each type of the plurality of types of data (e.g., medical, wellness, and/or training/performance) is assigned a different graphical affordance. Specifically, the body map generator 140 may analyze the stored MWT records for a particular subject and create a different aggregate for each set of MWT records that indicate the same body part and the same type of data (e.g., medical, wellness, or training/performance). For example, the body map generator 140 may aggregate together all MWT records for the particular subject (e.g., based on the header with the MWT system identifier) that indicate head as the body part (e.g., bodypart field stores a value of 20) and also indicate a data type of medical (e.g., type field stores a value of 2) to form a first aggregate. In addition, the body map generator 140 may aggregate all MWT records for the particular subject that indicate right arm as the body part (e.g., bodypart field stores a value of 1) and also indicate a data type of training/performance (e.g., type field stores a value of 1) to form a second aggregate. Further, the body map generator 140 may aggregate together all MWT records for the particular subject that indicate right buttock as the body part and also indicate a data type of medical (e.g., type field stores a value of 2) to form a third aggregate.

The body map generator 140 may then highlight body parts on the interactive computer-generated body map based on the type indicated in the created aggregates, wherein each type may be provided with a different graphical affordance. For example, medical data may be assigned a first shading, wellness data may be assigned a second shading, and training/performance data may be assigned a third shading. As such, and following the example above, the body map generator 140 may highlight the head, on the interactive computer-generated body map, with the first shading to indicate that the head has associated with medical data, e.g., medical MWT records of the first created aggregate for the particular subject. In addition, the body map generator 140 may highlight the right arm, on the interactive computer-generated body map, with the third shading to indicate that the right arm has associated training/performance data, e.g., training/performance MWT records of the second created aggregate for the particular subject. Further, the body map generator 140 may highlight the right buttock, on the interactive computer-generated body may, with the first shading to indicate that the right buttock has associated medical data, e.g., medical MWT records of the third created aggregate for the particular subject. As such, the body map generator 140 provides a visual indication on the interactive computer-generated body map as to what type of data is associated with which body parts.

If a plurality of created aggregates indicate the same body part (e.g., head) but indicate a different type of data, the body map generator 140 may implement any of a variety of different priority algorithms to determine how the body part should be highlighted on the interactive computer-generated body map. Specifically, and following the example above, the body map generator 140 may aggregate together all MWT records indicating head as the body part (e.g., bodypart field stores a value of 20) and also indicate a data type of wellness (e.g., type field stores a value of 3) to form a fourth aggregate. Under this circumstance, the first aggregate and the fourth aggregate are created for the same body part, e.g., head, but are different types of data, e.g., respectively medical and wellness, that are assigned different graphical affordances. As such, the body map generator 140 may implement a priority algorithm indicating that medical data is to be given priority over wellness data and training/performance data, and training/performance data is to be given priority over wellness data. As such, the body map generator 140 highlights the head, on the interactive computer-generated body map, with the first shading instead of highlighting the head with the third shading.

That interactive computer-generated body map may also include one or more body system filters 920A-920K. In response to the selection of a particular body system filter, e.g., user selection, the body map generator 140 may only utilize the MWT records indicating the selected body system to highlights body parts on the body map. As depicted in interactive computer-generated body map 900, all the body systems are selected. Following the above example, let it be assumed that all the MWT records that make up the second aggregate, e.g., indicate right arm as the body part and also indicate a data type of training/performance, also indicate that the affected body system is the muscular body system. Thus, and since the training/performance MWT records that make up the second aggregate indicate that the muscular body system is the affected body system, the body map generator 140 highlights the right arm, on the interactive computer-generated body map, with the third shading when the muscular body system filter 920B is toggled on (i.e., selected). However, if the muscular body system filter 920B is toggled off (i.e., deselected), the body map generator 140 excludes all information (e.g., MWT records in the created aggregates) that indicates that muscular body system as the affected body system. As such, and when the muscular body system filter 920B is toggled off, the body map generator 140 does not highlight the right arm and the right arm remains unshaded (e.g., neutral).

The interactive computer-generated body map 900, generated utilizing the created MWT records, provides a pictorial representation, of a subject's medical, wellness, training/performance history over time, where the body parts with the highest level of interest/activity are highlighted by shading, coloring, or any other graphical affordance.

In response to a selection, e.g., user selection, of a body part on the interactive computer-generated body map 900, the body map generator 140 may create and present (display) a listing of entries associated with the MWT records that make up the one or more aggregates represented by the body part.

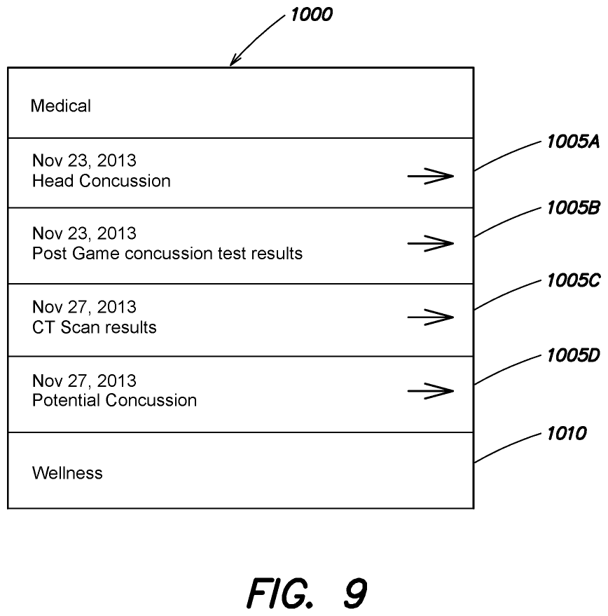

FIG. 9 is a schematic illustration of an example listing associated with the MWT records of an aggregate represented by a body part on the interactive computer-generated body map of a human being in accordance with the one or more embodiments described herein. The body map generator 140 may create and present listing 1000 in response to the selection of a body part, such as the head, on the interactive computer-generated body map. The listing 1000 includes a plurality of entries 1005A-1005D, each of which is associated with a different medical MWT records (e.g., type field stores value of 2 indicating medical and bodypart field stores a value of 20 indicating the head). The body map generator 140 may present the entries 1005A-1005D according to severity, with the entry having the highest severity being first in the listing. Alternatively, the body map generator 140 may present the entries 1005A-1005D utilizing any of a variety of different factors.

In the example provided, the first aggregate and the fourth aggregate are created for the same body part, e.g., head, but are different types of data, e.g., respectively medical and wellness. As such, and in response to a selection of wellness table 1010 from listing 1000, the body map generator 140 may create and present the listing of entries associated with the wellness MWT records that make up the fourth aggregate (e.g., type field stores value of 3 indicating wellness and bodypart field stores a value of 20 indicating the head). Although not depicted in FIG. 9, if another aggregate was created for the same body part, e.g., head, but was for training/performance data, the body map generator 140 would include in the listing 1000 a selectable training/performance tab such that the body map generator 140 may list the entries associated with the MWT records that make up the fourth aggregate.

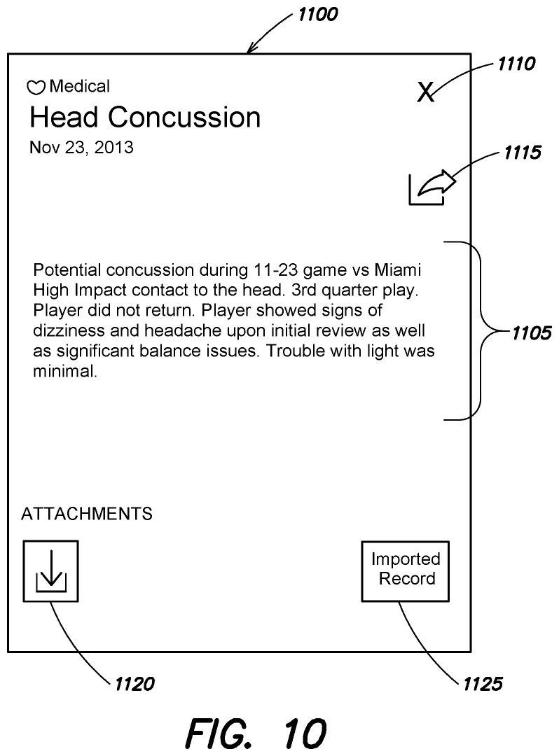

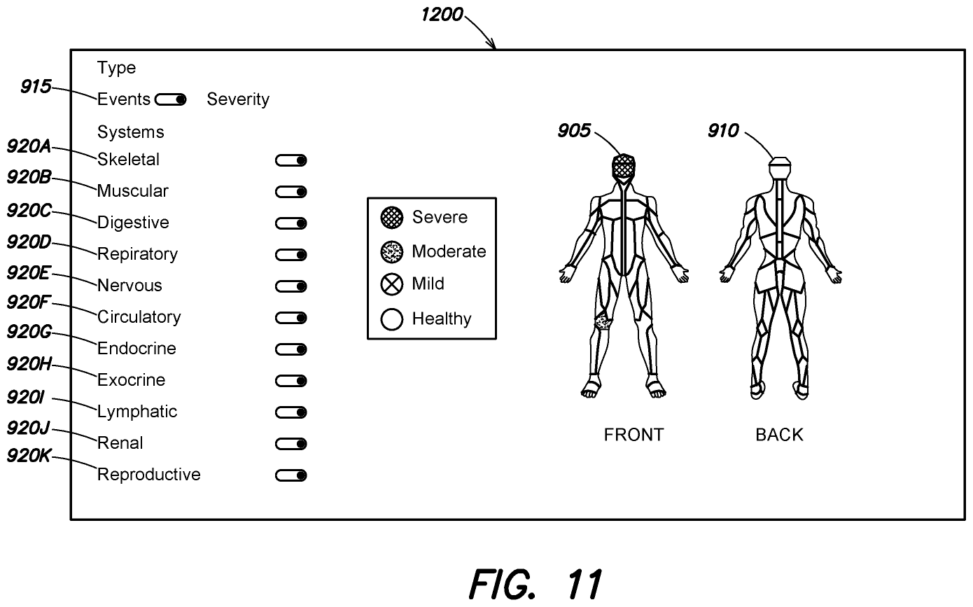

FIG. 10 is a schematic illustration of an entry from a listing associated with MWT records for a human being in accordance with one or more embodiments described herein. The entry 1100 corresponds to a particular MWT record from the listing in FIG. 9 (e.g., 1005A). The entry 1100 includes the brief description, the date, and a detailed description field 1105. The detailed description field 1105 may store the details for the MWT records. For this example, the detailed description may include the details regarding a head concussion incurred by a subject. The body map generator 140 may extract the detailed description from the associated MWT record or the corresponding imported record and store the detailed description in description field 1105. In response to a selection of “X” 1110, e.g., user selection, the body map generator 140 may revert back to back to the listing 1000. In response to a selection of button 1115, the body map 140 generator may provide a mechanism to forward the entry (e.g., MWT record) to a different user (e.g., doctor, specialist, etc.). Further, and in response to selection of attachment button 1120, the body map generator 140 may obtain and present, to a user, the attachment associated with the MWT record. In response to a selection of imported record button 1125, the body map generator 140 may obtain, from the one or more DB S 160, the linked imported record and present, to a user, the imported record for which the MWT record is created.

Advantageously, a user can investigate in real-time a subject's medical, wellness, and/or training performance history and the relationship between medical, wellness, and/or training performance data and different body parts/systems. Therefore, the subject can adapt his future behavior by obtaining a better understanding of the subject's medical, wellness, and/or training performance utilizing interactive computer-generated body map.

When the toggle 915 (FIG. 8) is set to severity, the interactive computer-generated body map is displayed by the MWT system 105 to highlight (e.g., shading, color, etc.) body parts of the human being to indicate severity of harm on the interactive computer-generated body map.

FIG. 11 is a schematic illustration of a computer display depicting an interactive computer-generated body map that highlights body parts of a human being based on severity of harm in accordance with one or more embodiment described herein. Specifically, the body map generator 140 may analyze the stored MWT records for a particular subject and create a different aggregate for each set of medical MWT records (e.g., type field stores a value of 2) that indicate the same body part. As such, there is at most one aggregate created for each body part. The body map generator 140 may then utilize any a variety of factors associated with each aggregate to highlight body parts based on severity of harm. For example, the severity of harm may be healthy, mild, moderate, or severe, and each severity of harm may be assigned a different shading, color, etc.

The body map generator 140 may determine a severity of harm for each body part utilizing the information from and associated with the one or more medical MWT records that make up the aggregate corresponding to the body part. The information may include, but is not limited to, the severity (e.g., impact field), date, and/or status indicated in the one or more medical MWT records that make up the aggregate corresponding to the body part. The body map generator 140 may then utilize the information with any a variety of different algorithm, to determine which severity of harm should be assigned to the body part.

For example, the algorithm utilized with the medical interactive computer-generated lifeline 605 (e.g., if a marking should be included and size of marking) may be utilized by the body map generator 140 to determine the severity of harm that should be assigned to the body part on the body map. Specifically, the algorithm implemented by the body map generator 140 may indicate that the body part should be highlighted with a first graphical affordance, indicating healthy, on the body map if all medical MWT record in the aggregate and associated with the body part indicates impact/severity that is not greater than 1. The algorithm may also indicate that the body part should be highlighted with a second graphical affordance, indicating mild severity of harm, on the body map if any medical MWT record in the aggregate and associated with the body part indicates an impact/severity that is at least 2 and not greater than 2. Further, the algorithm may indicate that the body part should be highlighted with a third graphical affordance, indicating moderate severity of harm, on the body map if any medical MWT record in the aggregate and associated with the body part indicates impact/severity that is at least 3 and not greater than 3. In addition, the algorithm may indicate that the body part should be highlighted with a fourth graphical affordance, indicating severe severity of harm, on the body map if any medical MWT record in the aggregate and associated with the body part indicates an impact/severity that is at least 4.

The algorithm described above is for illustrative purposes only, and it is expressly contemplated that the body map generator 140 may utilize any of a variety of different algorithms to determine the severity of harm for a body part utilizing information such as, but not limited to, the severity (e.g., impact field), date, and/or status indicated in the one or more medical MWT records that make up the aggregate corresponding to the body part.

The body map generator 140 highlights the head, on the interactive computer-generated body map on computer display 1200 of FIG. 11, with the fourth shading indicating severe severity of harm. Therefore, at least one medical MWT records of the aggregate corresponding to the head indicates an impact/severity that is at least 4. In addition, the body map generator 140 highlights the knee, on the interactive computer-generated body map on computer display 1200 of FIG. 11, with the third shading indicating moderate severity of harm. Therefore, the medical MWT records of the aggregate corresponding to the knee indicate an impact/severity that is at least 3 and not greater than 3. As such, the interactive computer-generated body map provides a pictorial representation, of a subject's medical history over time, where the body parts with the highest level of harm are highlighted by shading, coloring, or any other graphical affordance.

As described above with reference to FIG. 8, in response to the selection of a particular body system filter, e.g., user selection, the body map generator 140 may only utilize the MWT records indicating the selected body system to highlight body parts on the body map. As such, the body map generator 140 may highlight body parts based on severity and utilizing only the MWT records indicating the selected body system. In addition, and as described with reference to FIG. 9, in response to a selection, e.g., user selection, of a body part on the interactive computer-generated body map of FIG. 11, the body map generator 140 may create and present a listing of entries associated with the MWT records that make up the aggregate corresponding to the selected body. Further, and as described above with reference to FIG. 10, the body map generator 140 may present an entry from the listing based on a selection.

Advantageously, a user can investigate in real-time a subject's medical history based on severity and the relationship between medical data and different body parts/systems.

Although the interactive computer-generated lifelines of FIG. 5 and the interactive computer-generated body map of FIGS. 8 and 11 appear on different display, it is expressly contemplated that the interactive computer-generated lifelines and the interactive computer-generated body map may be on the same computer display. Advantageously, a user may be able to utilize the features or functions of the interactive computer-generated lifelines and features and functions of the interactive computer-generated body map together in a single display screen.

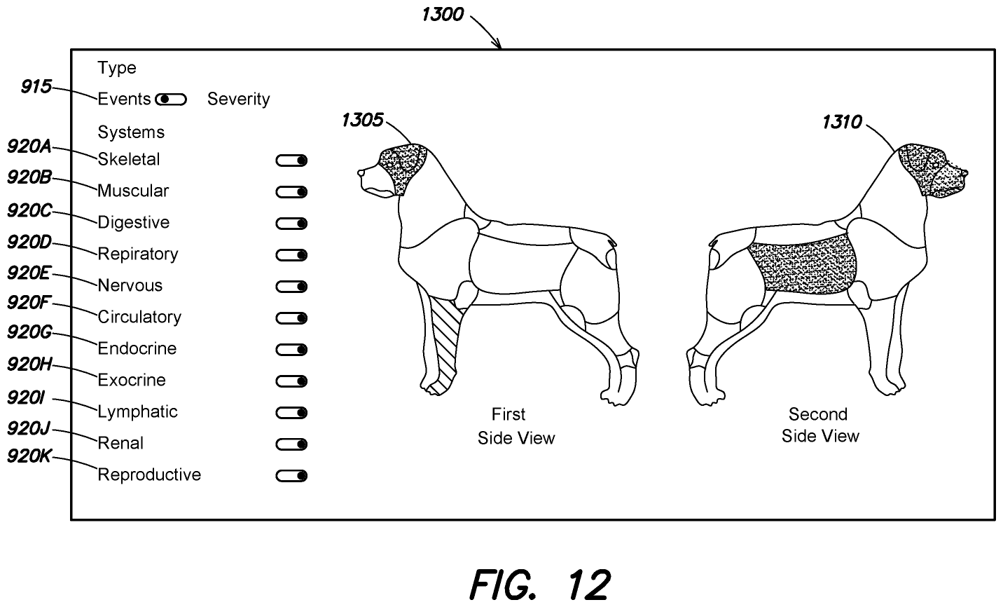

FIG. 12 is a schematic illustration of an example computer display depicting an interactive computer-generated body map that highlights body parts of an animal based on data type in accordance with one or more embodiment described herein. The interactive computer-generated body map, on computer display 1300, includes a first side view of a canine avatar 1305 and a second side view of a canine avatar 1310. The first side view of the canine avatar 1305 and the second side view of the canine avatar 1310 depict body parts associated with the canine anatomy. Particular body parts on the first side view of the canine avatar 1305 and the second side view of the canine avatar 1310 may be highlighted utilizing shading, coloring, or any of a variety of different graphical affordances.

As described above with reference to FIG. 8, when the toggle 915 is set to events, the body map generator 140 highlights body parts (e.g., shading, color, etc.) of the animal on the interactive computer-generated body map according to the type of data, where each type of the plurality of types of data (e.g., medical, wellness, and/or training/performance) is assigned a different graphical affordance. Specifically, the body map generator 140 may analyze the stored MWT records for a canine and create a different aggregate for each set of MWT records that indicate the same body part and the same type of data (e.g., medical, wellness, or training/performance). For example, the body map generator 140 may aggregate together all MWT records for the canine (e.g., based on the header with the MWT system identifier) that indicate head as the body part and also indicate a data type of medical (e.g., type field stores a value of 2) to form a first aggregate. In addition, the body map generator 140 may aggregate together all MWT records for the canine that indicate front left leg as the body part and also indicate a data type of training/performance (e.g., type field stores a value of 1) to form a second aggregate. Further, the body map generator 140 may aggregate together all MWT records for the canine that indicate right abdominal area as the body part and also indicate a data type of medical (e.g., type field stores a value of 2) to form a third aggregate.

The body map generator 140 may then highlight body parts of the animal on the interactive computer-generated body map based on the type indicated in the created aggregates, wherein each type may be provided with a different graphical affordance.

Specifically, and following the example above, the body map generator 140 may highlight the head of the canine, on the interactive computer-generated body map, with the first shading to indicate that the head has associated medical data, e.g., medical MWT records of the first created aggregate for the canine. In addition, the body map generator 140 may highlight the front left leg of the canine, on the interactive computer-generated body map, with the third shading to indicate that the front left leg has associated training/performance data, e.g., training/performance MWT records of the second created aggregate for the particular canine. Further, the body map generator 140 may highlight the right abdominal area of the canine, on the interactive computer-generated body may, with the first shading to indicate that the left abdominal area has associated medical data, e.g., medical MWT records of the third created aggregate for the canine. As such, the body map generator 140 provides a visual indication on the interactive computer-generated body map as to what type of data, e.g., MWT records, is associated with which body parts of the canine.

If a plurality of created aggregates indicate the same body part (e.g., head) of the animal but indicate a different type of data, the body map generator 140 may implement any of a variety of different priority algorithms to determine how the body part of the animal should be highlighted on the interactive computer-generated body map in a similar manner as described above with reference to FIG. 8.

The interactive computer-generated body map may also include one or more body system filters 920A-920K associated with the animal. In response to the selection of a particular body system filter, e.g., user selection, the body map generator 140 may only utilize the MWT records indicating the selected body system to highlight body parts on the body map in a similar manner as described above with reference to FIG. 8.

As such, interactive computer-generated body map 1300, generated utilizing the created MWT records, provides a pictorial representation, of an animal's (e.g., canine's) medical, wellness, training/performance history over time, where the body parts of the animal with the highest level of interest/activity are highlighted by shading, coloring, or any of a variety of different graphical affordances.

In response to a selection, e.g., user selection, of a body part of the animal on the interactive computer-generated body map 1300, the body map generator 140 may create and present (display) a listing of entries associated with the MWT records that make up the one or more aggregates represented by the body part of the animal.

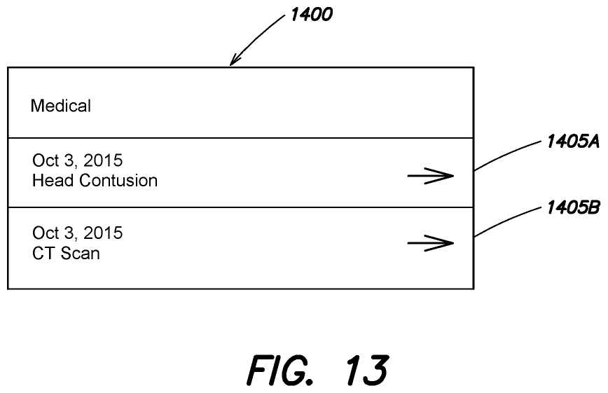

FIG. 13 is a schematic illustration of an example listing associated with the MWT records of an aggregate represented by a body part on the interactive computer-generated body map of an animal in accordance with the one or more embodiments described herein. The body map generator 140 may create and present listing 1400 in response to the selection of a body part, such as the head, on the interactive computer-generated body map. The listing 1400 includes a plurality of entries 1405A and 1405B, each of which is associated with a different medical MWT records (e.g., type field stores value of 2 indicating medical and bodypart field stores a value indicating the head of the canine). The body map generator 140 may present the entries 1405A and 1405B according to severity, with the entry having the highest severity being first in the listing. Alternatively, the body map generator 140 may present the entries 1405A and 1405B utilizing any of a variety of different factors.

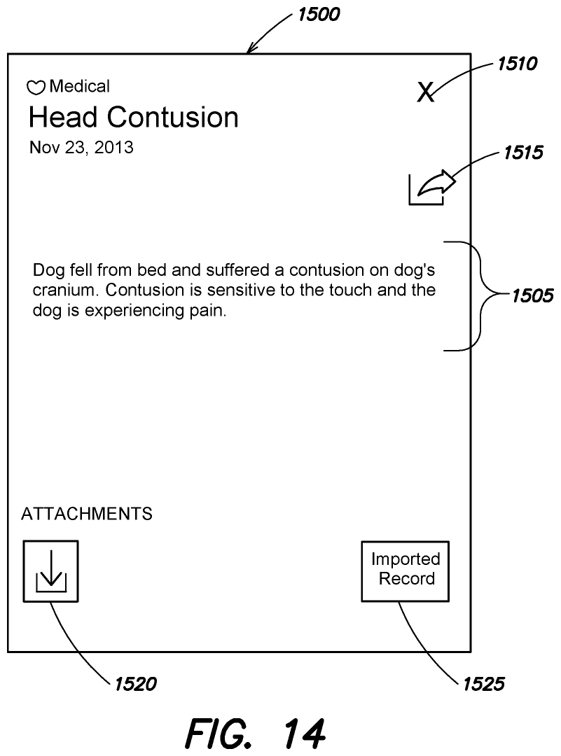

FIG. 14 is a schematic illustration of an entry from a listing associated with MWT records for an animal in accordance with one or more embodiments described herein. The entry 1500 corresponds to a selected MWT record from the listing in FIG. 13 (e.g., 1405A). The entry 1500 includes the brief description, the date, and a detailed description field 1505. The detailed description field 1505 may store the details for the MWT record. For this example, the detailed description may include the details regarding a head contusion incurred by the canine, e.g., that the dog fell off the bed and obtained a contusion on his cranium. The body map generator 140 may extract the detailed description from the associated MWT record or the corresponding imported record and store the detailed description in description field 1505. In response to a selection of “X” 1510, e.g., user selection, the body map generator 140 may close entry 1500 and revert back to the listing 1400. In response to a selection of button 1515, the body map 140 generator may provide a mechanism to forward the entry (e.g., MWT record) to a different user (e.g., doctor, specialist, etc.). Further, and in response to selection of attachment button 1520, the body map generator 140 may obtain and present, to a user, the attachment associated with the MWT record. In response to a selection of imported record button 1525, the body map generator 140 may obtain, from DBs 160, the linked imported record and present, to a user, the imported record for which the MWT record is created.

Advantageously, the user can investigate in real-time the animal's medical, wellness, and/or training performance history and the relationship between medical, wellness, and/or training performance data and different body parts/systems. Therefore, the future behavior of the animal may be adapted by obtaining a better understanding of the animal's medical, wellness, and/or training performance utilizing interactive computer-generated body map.

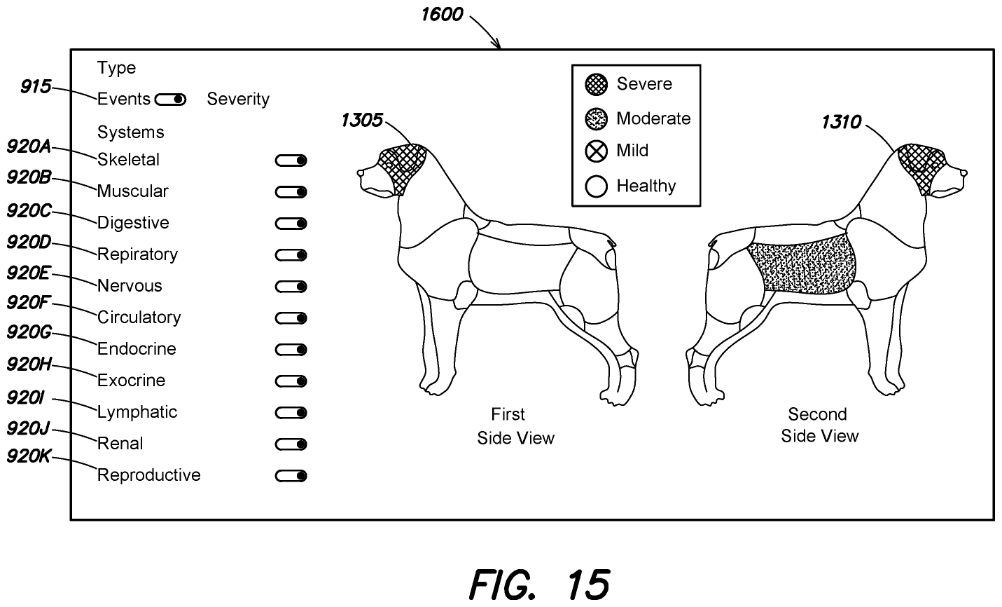

When the toggle 915 is set to severity, the interactive computer-generated body map is displayed to highlight (e.g., shading, color, etc.) body parts of the animal to indicate severity of harm on the interactive computer-generated body map. FIG. 15 is a schematic illustration of a computer display depicting an interactive computer-generated body map that highlights body parts of an animal based on severity of harm in accordance with one or more embodiment described herein. Specifically, the body map generator 140 may analyze the stored MWT records for a particular animal and create a different aggregate for each set of medical MWT records (e.g., type field stores a value of 2) that indicate the same body part. As such, there is at most one aggregate created for each body part of the animal. The body map generator 140 may then utilize any a variety of factors associated with each aggregate to highlight body parts of the animal based on severity of harm. For example, the severity of harm may be healthy, mild, moderate, or severe, and each severity of harm may be assigned a different shading, color, etc.

For example, the algorithm utilized with the medical interactive computer-generated lifeline 605 (e.g., if a marking should be included and size of marking) may be utilized by the body map generator 140 to determine the severity of harm that should be assigned to the body parts on the body map for the animal. For example, the body map generator 140 highlights the head of the canine, on the interactive computer-generated body map on the computer display 1600 of FIG. 15, with the fourth shading indicating severe severity of harm. Therefore, at least one medical MWT records of the aggregate corresponding to the head indicates an impact/severity that is at least 4. In addition, the body map generator 140 highlights the front left leg of the canine, on the interactive computer-generated body map on the computer display 1600 of FIG. 15, with the third shading indicating moderate severity of harm. Therefore, the medical MWT records of the aggregate corresponding to the front left leg indicate an impact/severity that is at least 3 and not greater than 3. As such, the interactive computer-generated body map provides a pictorial representation, of an animal's medical history over time, where the body parts of the animal with the highest level of harm are highlighted by shading, coloring, or any of a variety of different graphical affordances.

As described above with reference to FIGS. 12, in response to the selection of a particular body system filter, e.g., user selection, the body map generator 140 may only utilize the MWT records indicating the selected body system to highlight body parts of the animal on the body map. As such, the body map generator 140 may highlight body parts based on severity and utilizing only the MWT records indicating the selected body system. In addition, and as described with reference to FIGS. 12 and 13, in response to a selection, e.g., user selection, of a body part on the interactive computer-generated body map of FIG. 15, the body map generator 140 may create and present a listing of entries associated with the MWT records that make up the aggregate corresponding to the selected body part. Further, and as described above with reference to FIGS. 14, the body map generator 140 may present an entry from the listing based on a selection.

Advantageously, a user can investigate in real-time an animal's medical history based on severity and the relationship between medical data and different body parts/systems.

Although the interactive computer-generated lifelines of FIG. 5 and the interactive computer-generated body map of FIGS. 12 and 15 appear on different display, it is expressly contemplated that the interactive computer-generated lifelines and the interactive computer-generated body map may be on the same computer display.

Advantageously, a user may be able to utilize the features or functions of the interactive computer-generated lifelines and features and functions of the interactive computer-generated body map together in a single display screen.

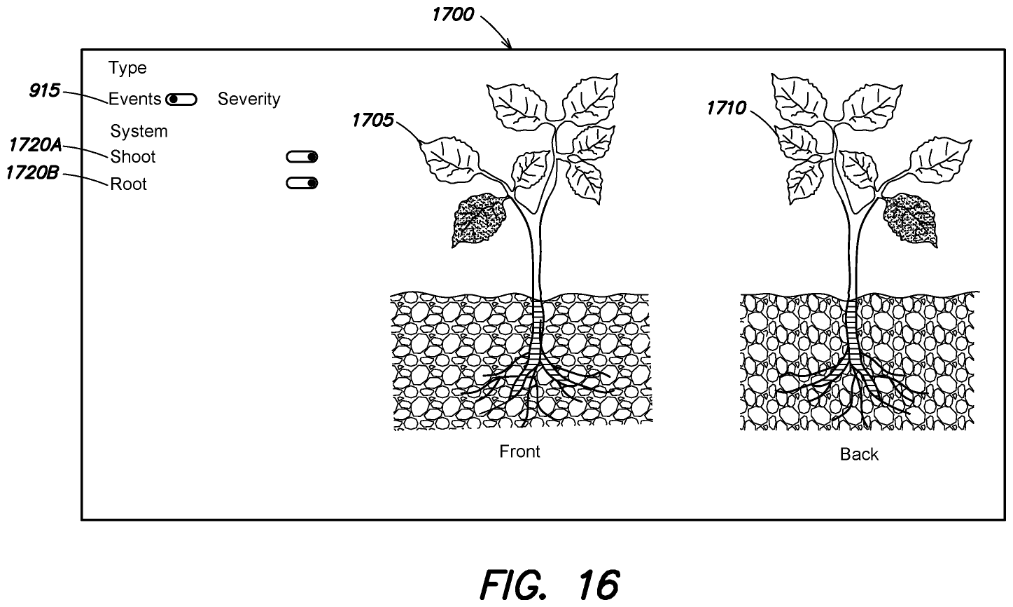

FIG. 16 is a schematic illustration of an example computer display depicting an interactive computer-generated body map that highlights body parts of a plant based on data type in accordance with one or more embodiment described herein. The interactive computer-generated body map, on computer display 1700, includes a front view of a plant avatar 1705 and a back view of a plant avatar 1710. The front view of the plant avatar 1705 and the back view of the plant avatar 1710 depict body parts associated with the anatomy of a plant. Particular body parts on the first side view of the plant avatar 1705 and the back view of the plant avatar 1710 may be highlighted utilizing shading, coloring, or any of a variety of different graphical affordances.

As described above, when the toggle 915 is set to events, the body map generator 140 highlights body parts (e.g., shading, color, etc.) of the plant on the interactive computer-generated body map according to the type of data, where each type of the plurality of types of data (e.g., medical, wellness, and/or training/performance) is assigned a different graphical affordance. Specifically, the body map generator 140 may analyze the stored MWT records for a particular plant and create a different aggregate for each set of MWT records that indicate the same body part and the same type of data (e.g., medical, wellness, or training/performance). For example, the body map generator 140 may aggregate together all MWT records for the plant (e.g., based on the header with the MWT system identifier) that indicate a particular leaf (where each leaf has a different associated identifier) as the body part and also indicate a data type of medical (e.g., type field stores a value of 2) to form a first aggregate. In addition, the body map generator 140 may aggregate together all MWT records for the plant that indicate roots as the body part and also indicate a data type of wellness to form a second aggregate (e.g., type field stores a value of 3).

The body map generator 140 may then highlight body parts of the plant on the interactive computer-generated body map based on the type indicated in the created aggregates, wherein each type may be provided with a different graphical affordance.

Specifically, the body map generator 140 may highlight the particular leaf of the plant, on the interactive computer-generated body map, with the first shading to indicate that the particular leaf has associated medical data, e.g., medical MWT records of the first created aggregate for the plant. In addition, the body map generator 140 may highlight the roots, on the interactive computer-generated body map, with the second shading to indicate that the roots have associated wellness data, e.g., wellness MWT records of the second created aggregate for the plant. As such, the body map generator 140 provides a visual indication on the interactive computer-generated body map as to what type of data is associated with which body parts of the plant.

If a plurality of created aggregates indicate the same body part (e.g., the same particular leaf) but indicate a different type of data, the body map generator 140 may implement any of a variety of different priority algorithms to determine how the body part of the plant should be highlighted on the interactive computer-generated body map in a similar manner as described above with reference to FIG. 8.

That interactive computer-generated body map may also include one or more system filters 1720A and 1720B. For example, system filter 1720A may be associated with the shoot system of the plant (e.g., above ground and includes the organs such as leaves, buds, stems, flowers, and/or fruits), while system filter 1720B may be associated with the root system of the plant. In response to the selection of a particular system filter, e.g., user selection, the body map generator 140 may only utilize the MWT records indicating the selected system to highlight body parts on the body map associated with the plant in a similar manner as described above with reference to FIG. 8.

As such, interactive computer-generated body map 1700, generated utilizing the created MWT records, provides a pictorial representation, of a plant's medical, wellness, training/performance history over time, where the body parts of the plant with the highest level of interest/activity are highlighted by shading, coloring, or any of a variety of different graphical affordances.

In response to a selection, e.g., user selection, of a body part of the plant on the interactive computer-generated body map 1700, the body map generator 140 may create and present (display) a listing of entries associated with the MWT records that make up the one or more aggregates represented by the body part of the plant.

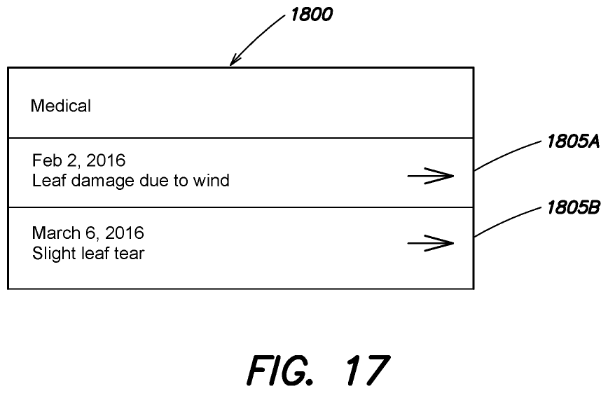

FIG. 17 is a schematic illustration of an example listing associated with the MWT records of an aggregate represented by a body part on the interactive computer-generated body map of a plant in accordance with the one or more embodiments described herein. The body map generator 140 may create and present listing 1800 in response to the selection of a body part, such as a particular leaf, on the interactive computer-generated body map. The listing 1800 includes a plurality of entries 1805A and 1805B, each of which is associated with a different medical MWT records (e.g., type field stores value of 2 indicating medical and body part field stores a value associated with the particular leaf). The body map generator 140 may present the entries 1805A and 1805B according to severity, with the entry having the highest severity being first in the listing. Alternatively, the body map generator 140 may present the entries 1805A and 1805B utilizing any of a variety of different factors.

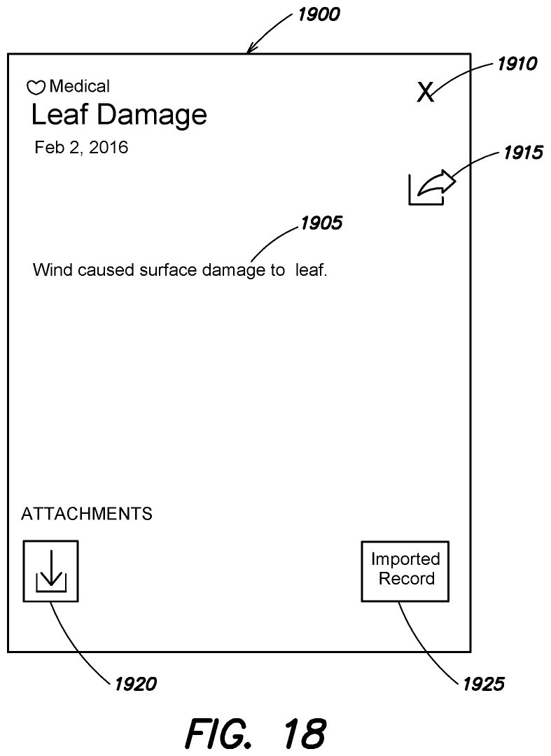

FIG. 18 is a schematic illustration of an entry from a listing associated with MWT records for a plant in accordance with one or more embodiments described herein. The entry 1900 corresponds to a selected MWT record from the listing in FIG. 17 (e.g., 1805A). The entry 1900 includes the brief description, the date, and a detailed description field 1905. The detailed description field 1905 may store the details for the MWT records. For this example, the detailed description may include the details regarding the damage incurred by the particular leaf of due to wind. The body map generator 140 may extract the detailed description from the associated MWT record or the corresponding imported record and store the detailed description in description field 1905. In response to a selection of “X” 1910, e.g., user selection, the body map generator 140 may close entry 1900 and revert back to back to the listing 1800. In response to a selection of button 1915, the body map 140 generator may provide a mechanism to forward the entry (e.g., MWT record) to a different user (e.g., doctor, specialist, etc.). Further, and in response to selection of attachment button 1920, the body map generator 140 may obtain and present, to a user, the attachment associated with the MWT record. In response to a selection of imported record button 1925, the body map generator 140 may obtain, from DBs 160, the linked imported record and present, to a user, the imported record for which the MWT record is created.

Advantageously, the user can investigate in real-time a plants medical wellness, and/or training/performance history and the relationship between medical, wellness, and/or training/performance for different parts/systems. Therefore, the future behavior associated with plant can be adapted by obtaining a better understanding of the plant medical, wellness, and/or training/performance utilizing interactive computer-generated body map.

When the toggle 915 is set to severity, the interactive computer-generated body map is displayed to highlight (e.g., shading, color, etc.) body parts of the plant to indicate severity of harm on the interactive computer-generated body map.

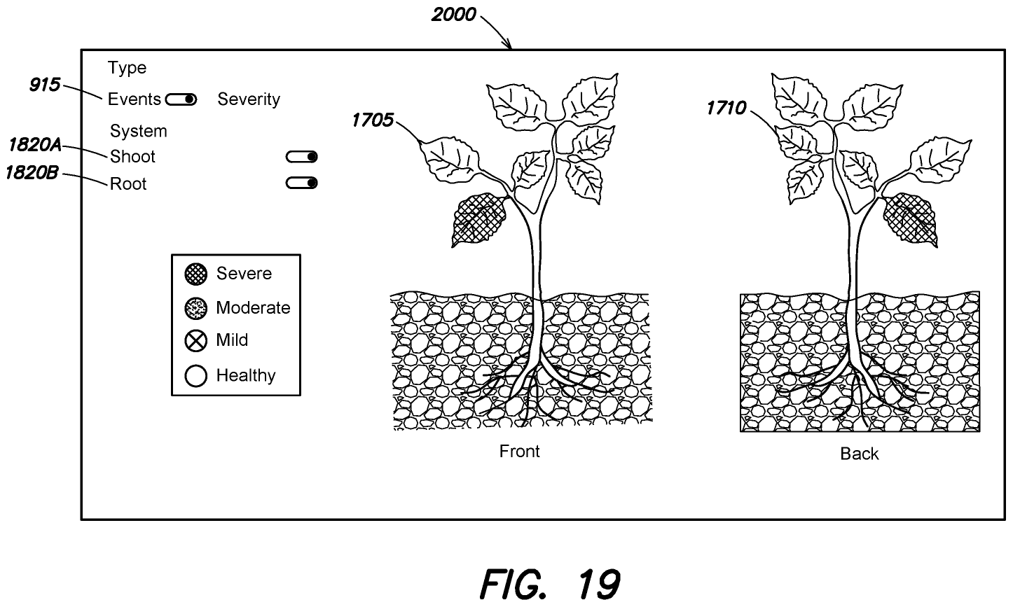

FIG. 19 is a schematic illustration of a computer display depicting an interactive computer-generated body map 2000 that highlights parts of a plant based on severity of harm in accordance with one or more embodiment described herein. Specifically, the body map generator 140 may analyze the stored MWT records for the plant and create a different aggregate for each set of medical MWT records (e.g., type field stores a value of 2) that indicate the same body part. As such, there is at most one aggregate created for each body part of the plant. The body map generator 140 may then utilize any a variety of factors associated with each aggregate to highlight body parts of the plant based on severity of harm. For example, the severity of harm may be healthy, mild, moderate, or severe, and each severity of harm may be assigned a different shading, color, etc.

For example, the algorithm utilized with the medical interactive computer-generated lifeline 605 (e.g., if a marking should be included and size of marking) may be utilized by the body map generator 140 to determine the severity of harm that should be assigned to the body parts on the body map for the plant. For example, the body map generator 140 highlights the particular leaf of the plant, on the interactive computer-generated body map 2000 of FIG. 19, with the fourth shading indicating severe severity of harm. Therefore, at least one medical MWT records of the aggregate corresponding to the particular leaf indicates an impact/severity that is at least 4. As such, the interactive computer-generated body map 2000 provides a pictorial representation, of a plant's medical history over time, where the body parts of the plant with the highest level of harm are highlighted by shading, coloring, or any of a variety of different graphical affordances.

As described above with reference to FIGS. 16, in response to the selection of a particular system filter, e.g., user selection, the body map generator 140 may only utilize the MWT records indicating the selected system to highlight body parts of the plant on the body map. As such, the body map generator 140 may highlight body parts of the plant based on severity and utilizing only the MWT records indicating the selected system. In addition, in response to a selection, e.g., user selection, of a body part on the interactive computer-generated body map 2000 of FIG. 19, the body map generator 140 may create and present a listing of entries associated with the MWT records that make up the aggregate corresponding to the selected body part of the plant in a similar manner as described with reference to FIG. 17. Further, the body map generator 140 may present an entry from the listing based on a selection in a similar manner as described with reference to FIG. 18.

Advantageously, a user can almost instantaneously investigate a plant's medical, history based on severity and the relationship between medical data and different parts/systems.

In addition, the MWT system 105 may link the interactive computer-generated body map may to the interactive computer-generated lifeline. For example, a user may utilize the slider 655 or other mechanism to focus the interactive computer-generated lifeline on a particular time period. In response, the MWT system 105 may only utilize the MWT records within that time period to mark the body parts on the interactive computer-generated body map.

Although FIGS. 16-19 refer to generating interactive computer displays for a single plant, it is expressly contemplated that the MWT system 105 may generate interactive computer displays for vegetation, i.e., a collection of plants, in a similar manner as described herein. For example, the MWT system 105 may generate a body map for vegetation, e.g., a portion of a vineyard, that provides a pictorial representation, of the vegetation's medical, wellness, training/performance history over time, where areas/regions of the vegetation with the highest level of interest/activity are highlighted by shading, coloring, or any of a variety of different graphical affordances.

In an embodiment, a user registered with the MWT system 105 may want to provide other users, e.g., doctor, trainer, etc., with access to his/her data and the functions of the MWT system 105. As such, the security unit 150 may create and maintain a permissions mapping.

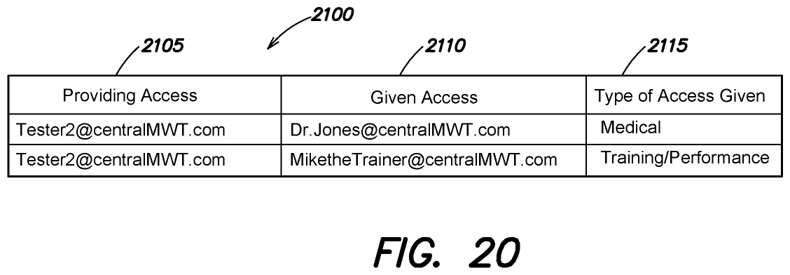

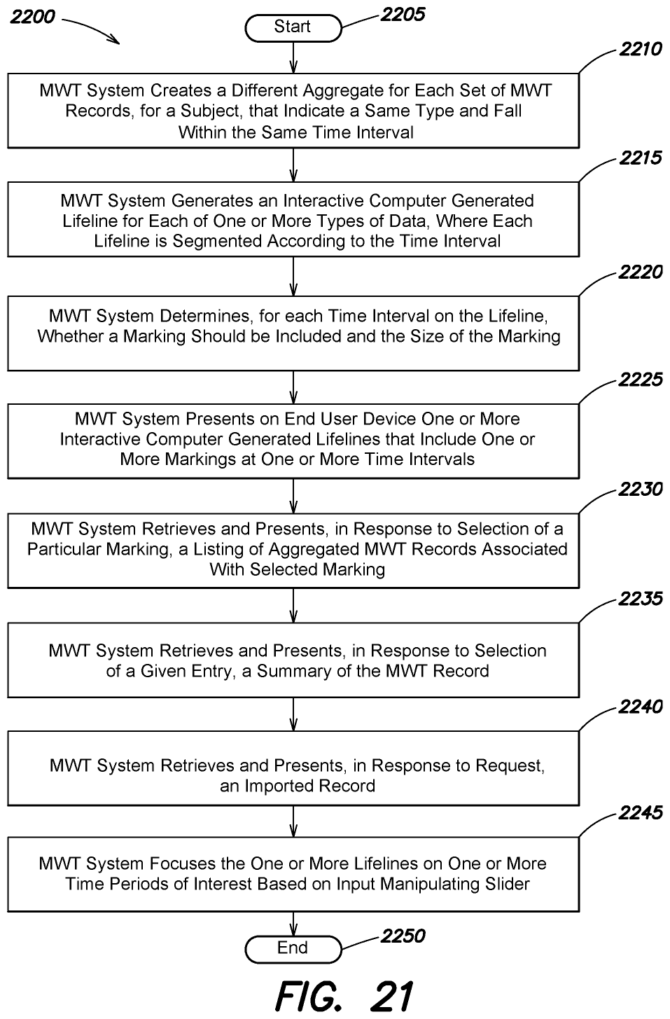

FIG. 20 a schematic illustration of a mapping in accordance with one or more embodiments described herein. The security unit 150 may store, within permissions mapping 2100, associations between first users having data and different users being provided access, by the first users, to the first user's data. Specifically, permissions mapping 2100 includes a first section 2105, such as a column or a row, which stores the first MWT system identifier associated with a first user that is providing the access. In addition, permissions mapping 2100 includes a second section 2110, such a column or row, which stores one or more different MWT system identifiers associated with one or more different users who have been given access to the first user's data. Further, permissions mapping 2100 includes a type section 2115, such as a column or row, which stores the type of permissions the one or more different users has access to the first' user's data.

For example, a first user, Tester2, may utilize an end user device 110 to access one or more webpages associated with the MWT system 105, and provide user credentials (e.g., MWT system identifier) to gain access to the user's MWT system account. The first user may utilize the one or more client-facing UIs to input information associated with his/her doctor, e.g., Dr. Jones, to provide his/her doctor with particular permissions, e.g., permission to the user's medical data. As such, the security unit 150 stores the Tester 2 in first section 2105. In addition, the security unit 150 stores Dr. Jones in section 2110, and the type of access, e.g., medical, provided to Dr. Jones in section 2115. In addition, the first user may utilize the one or more client-facing UIs to input information associated with his/her trainer, e.g., Mike the Trainer, to provide his/her training with particular permissions, e.g., permission to the user's training/performance data. As such, the security unit 150 stores Tester2 in section 2105. In addition, the security unit 150 stores Mike the Trainer in section 2110, and the type of access, e.g., training/performance, provided to Mike the Trainer in section 2115.

Subsequently, Dr. Jones may then utilize an end user device 110 to access one or more webpages associated with the MWT system 105, and provide user credentials (e.g., MWT system identifier for the different user) to gain access to the MWT system 105. The security unit 150 may then access the permissions mapping 2100 to determine that Dr. Jones has been given permission to Tester2's medical data. As such, the MWT system 105 may generate and display the medical interactive computer-generated lifeline to Dr. Jones as described above with reference to FIGS. 5-7, without generating and displaying the wellness and training/performance interactive computer-generated lifelines. In addition, the MWT system 105 may generate and display the interactive computer-generated body map, as described above with reference to FIG. to FIGS. 8-11, utilizing only the medical MWT records.

Similarly, the MWT system 105 may generate and display the training/performance interactive computer-generated lifeline to Mike the Trainer as described above with reference to FIGS. 5-7, without displaying and generating the wellness and medical interactive computer-generated lifelines. In addition, the MWT system 105 may generate and display the interactive computer-generated body map, as described above with reference to FIG. to FIGS. 8-11, utilizing only the training/performance MWT records.

FIG. 21 is a schematic illustration of a flow diagram of an example method for generating and operating an interactive computer-generated lifeline according to one or more embodiments described herein. The procedure 2200 starts at step 2205 and continues at step 2210 where the MWT system 105 creates a different aggregate (e.g., a grouping) for each set of MWT records, for a subject (e.g., human being, animal, plant, vegetation, etc.), that indicate a same type (e.g., medical, wellness, or training/performance) and fall within the same time interval. For example, the lifeline generator 130 may aggregate together all medical MWT records (e.g., type field stores a value of 2) for a particular subject (e.g., based on the header with the same MWT system identifier) that fall within the year of 1990 to form a first aggregate of medical MWT records. In addition, the lifeline generator 130 may aggregate together all medical MWT records for the particular subject that fall within the year of 1991 to form a second aggregate of medical MWT records, and so forth.

The procedure continues to step 2215 and the MWT system 105 generates an interactive computer-generated lifeline for each of one or more types of data, where each interactive computer-generated lifeline is segment/organize according to the time interval. Each lifeline for the different type of data may be visually differentiated from each other utilizing shading, coloring, or any of a variety of different graphical affordances. For example, and with reference to FIG. 5, there may be a medical interactive computer-generated lifeline 605, a wellness interactive computer-generated lifeline 610, and a training/performance interactive computer-generated lifeline 615. The illustrative interactive computer-generated lifelines, e.g., 605, 610, and 615, of computer display 600, are segmented and organized by the lifeline generator 130 according to a yearly time interval. The lifelines may be a longitudinal, vertical, diagonal, or any shaped graphs.

The procedure continues to step 2220 and the MWT system 105 determines, for each time interval on the interactive computer-generated lifeline, whether a marking should be included at the time interval and the size of the marking. For example, the lifeline generator 130 may analyze the impact/severity indicated and/or other information in each created aggregate and utilize any of a variety of different algorithms to determine if a marking should be included at the time interval.

The procedure continues to step 2225 and the MWT system 105 presents on the end user device the one or more interactive computer-generated lifelines that include one or more markings at one or more time intervals. The procedure continues to step 2230 and the MWT system retrieves and presents, in response to a selection of a particular marking on the lifeline, a listing of the aggregated MWT records associated with the selected marking. The procedure continues to step 2235 and the MWT system retrieves and presents, in response to the selection of a given entry from the list, a summary of the MWT record associated with the given entry. The procedure continues to step 2240 and the MWT system 105 retrieves and presents, in response to a request for additional detail, the imported record or one or more portions thereof that are associated with the given try. The procedure continues to step 2245 and the MWT system focuses the one or more lifelines on one or more time periods of interest based on input manipulating a slider or other UI element. The procedure ends at step 2250.

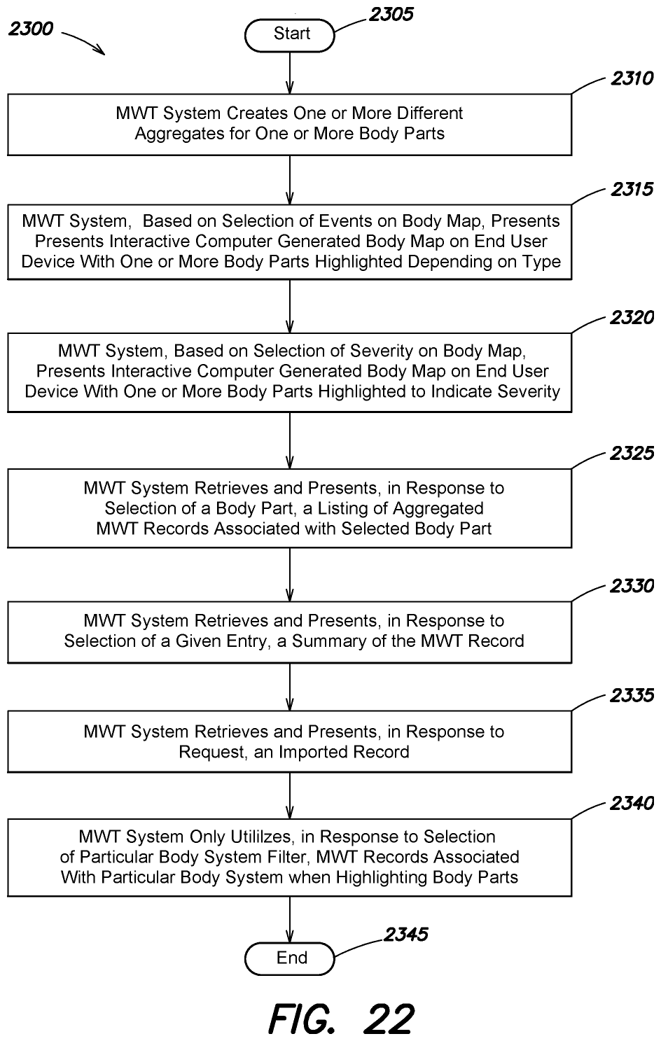

FIG. 22 is a schematic illustration of a flow diagram of an example method for generating and operating an interactive computer-generated body map according to one or more embodiments described herein. The procedure 2300 starts at step 2305 and continues at step 2310 where the MWT system 105 creates one or more different aggregate (e.g., a grouping) for one or more parts. Specifically, the MWT system 105 may create a different first aggregate from the MWT records, for a particular subject, that indicate the same body part (e.g., body part of a human's body, body part of an animal's body, body part of a plant, region of vegetation, etc.) and the same type (e.g., medical, wellness, or training/performance). In addition, the MWT system may create a different second aggregate from the medical MWT records (e.g., type is a value of 2) for the particular subject that indicate the same body part.

The procedure continues to step 2315 and the MWT system 105, based on selection of events on the body map, presents the interactive computer-generated body map on the end user device with one or more body parts highlighted depending on type. Specifically, the body map generator 140 may highlight one or more body parts indicated in the MWT records of the first aggregate based on the type indicated in the MWT records of the same first aggregate, where each type (e.g., medical, wellness, and/or training/performance) is assigned a different graphical affordance.

The procedure continues to step 2320 and the MWT system 105, based on selection of severity on the body map, presents the interactive computer-generated map on the end user device with one or more body parts that are highlighted to indicate a severity level (e.g., healthy, mild, moderate, severe). Specifically, the body map generator 140 may highlight one or more body parts indicated in the MWT records of a second aggregate based on the severity and/or other information (e.g., whether a condition is active or not) indicated in the MWT records of the same second aggregate. As such, the MWT system 105 may highlight body parts based on the severity level of the MWT records determined for the body parts.

The procedure continues to step 2325 and the MWT system 105 retrieves and presents, in response to a selection of a particular body part on the body map, a listing of the aggregated MWT records associated with the selected body part. The procedure continues to step 2330 and the MWT system 105 retrieves and presents, in response to the selection of a given entry from the list, a summary of the MWT record associated with the given entry. The procedure continues to step 2335 and the MWT system 105 retrieves and presents, in response to a request for additional detail, the imported record or one or more portions thereof that are associated with the given try. The procedure continues to step 2340 and the MWT system 105 only utilizes, in response to the selection of a particular body system filter on the body map, the MWT records associated with the selected body system when highlighting body parts on the body part as described at steps 2315 and 2320. The procedure ends at step 2345.

According to one or more embodiments described herein, health, wellness, and/or performance/training records may be evaluated and analyzed by the MWT system 105 to produce a readiness index value that may indicate a subject's readiness to exercise, train, and/or complete. In addition, the readiness index value may be calculated for a subject's health with respect to a “norm” such that the readiness index value may indicate whether the subject's health is improving, worsening, or staying the same. Thus, the readiness index allows for the selection of best training and best-care pathways to improve one or more care providers ability to determine the subject's readiness for exercise, training, competition and/or health.

It should be understood that a wide variety of adaptations and modifications may be made to the techniques described herein. In general, functionality may be implemented in software, hardware or various combinations thereof. Software implementations may include electronic device-executable instructions (e.g., computer-executable instructions) stored in a non-transitory electronic device-readable medium (e.g., a non-transitory computer-readable medium), such as a volatile memory, a persistent storage device, or other tangible medium. Hardware implementations may include logic circuits, application specific integrated circuits, and/or other types of hardware components. Further, combined software/hardware implementations may include both electronic device-executable instructions stored in a non-transitory electronic device-readable medium, as well as one or more hardware components. Above all, it should be understood that the above description is meant to be taken only by way of example.

1. A system, comprising:
a processor when executed configured to:
extract, based on an implementation of natural language processing, one or more keywords from each of a plurality of imported records associated with a subject,
determine, utilizing the extract keywords and the imported records, information for each of the imported records, wherein the information includes at least a type associated with the imported record, an affected body system associated with the imported record, an affected body part associated with the imported record, an impact level associated with the imported record, and an indication whether a condition, associated with the imported record, is active or not;
generate, utilizing the information determined for each of the plurality of imported records for the subject, one or more interactive computer-generated displays that provide a pictorial representation of the subject's medical history, wellness, training condition, and/or performance condition over time.

2. The system of claim 1, wherein the plurality of imported of records includes at least one of medical records including medical data, wellness records including wellness data, and performance records including training and/or performance data.

3. The system of claim 1, wherein the plurality of imported record includes structured data and unstructured data, and the structured data includes one or more codes that are associated with at least one of System Nomenclature of Medicine-Clinical Terms and International Classification of Diseases, Tenth Revision, Clinical Modification.

4. The system of claim 1, wherein the subject is one of a human being, an animal, plant, or vegetation.

5. The system of claim 1, wherein the processor is further configured to:
generate a medical interactive computer-generated lifeline for the subject utilizing the information determined for medical imported records of the subject, wherein one or more markings are included at one or more positions on the medical interactive computer-generated lifeline to represent a severity of harm, and each position is associated with a different time interval on the medical interactive computer-generated lifeline.

6. The system of claim 5, wherein the one or more markings include a first marking that is a first size and at a first time interval on the medical interactive computer-generated lifeline and a second marking that is a second size and at a second time interval on the medical interactive computer-generated lifeline, and wherein a first severity of harm represented by the first marking is different than a second severity of harm represented by the second marking.

7. The system of claim 6, wherein the processor is further configured to:
display, in response to receiving a selection of the first marking, a listing of entries corresponding to the medical imported records having a date that falls within first time interval.

8. The system of claim 7, wherein the processor is further configured to:
display, in response to receiving a selection a of a particular entry in the listing, selected information associated with the particular entry, wherein the particular entry corresponds to a particular medical imported record having the date that falls within the time interval.

9. The system of claim 1, wherein the processor is further configured to:
generate a wellness interactive computer-generated lifeline for the subject utilizing the information determined for wellness imported records of the subject, wherein one or more markings are included at one or more positions on the wellness interactive computer-generated lifeline to represent a wellness quality, and each position is associated with a different time interval on the wellness interactive computer-generated lifeline.

10. The system of claim 9, wherein the one or more markings include a first marking that is a first size and at a first time interval on the wellness interactive computer-generated lifeline and a second marking that is a second size and at a second time interval on the wellness interactive computer-generated lifeline, and wherein a first wellness quality represented by the first marking is different than a second wellness quality represented by the second marking.

11. The system of claim 1, wherein the processor is further configured to:
generate a training and/or performance interactive computer-generated lifeline for the subject utilizing the information determined for training and/or performance imported records of the subject, wherein one or more markings are included at one or more positions on the training and/or performance interactive computer-generated lifeline to represent a training and/or performance quality, and each position is associated with a different time interval on the training and/or performance interactive computer-generated lifeline.

12. The system of claim 11, wherein the one or more markings include a first marking that is a first size and at a first time interval on the training and/or performance interactive computer-generated lifeline and a second marking that is a second size and at a second time interval on the training and/or performance interactive computer-generated lifeline, and wherein a first training and/or performance quality represented by the first marking is different than a second training and/or performance quality represented by the second marking.

13. The system of claim 1, wherein the processor is further configured to:
generate an interactive computer-generated body map for the subject utilizing the particular determined for each of the plurality of imported records for the subject,
wherein one or more body parts on the interactive computer-generated body map are highlighted to indicate severity of harm body parts based on at least the first impact identifier of the information determined for medical imported records of the subject.

14. The system of claim 1, wherein the processor is further configured to:
generate an interactive computer-generated body map for the subject utilizing the information determined for the imported records of the subject,
wherein the one or more body parts on the interactive computer-generated body map are highlighted, utilizing the information determined for the imported records of the subject, to indicate which of the one or more body parts are associated with medical data, wellness data, or training and/or performance data.

15. A system, comprising:
a processor when executed configured to:
import a plurality of records, for a subject, from a data source and/or an end user device;
create, based on natural language processing and machine learning, an output record for each of the plurality of imported records, where each output record includes a type identifier for a type of the imported record, a body system identifier for an affected body system, a body part identifier for an affected body part, an impact identifier for an impact level, an is-active identifier for whether a condition is active or not, and a date identifier identifying a particular date;
create a first aggregate for a first set of the records, wherein each output record of the first aggregate includes the date identifier identifying the particular date that falls within a first time interval;
create a second aggregate for a second set of the records, wherein each output record of the second aggregate includes the date identifier identifying the particular date that within a second time interval; and
generate an interactive computer-generated lifeline for the subject that provides a pictorial representation of the user's medical history, wellness, or training over time,
wherein a first marking is included at a first position, corresponding to the first time interval, on interactive computer-generated lifeline to represent the first aggregate, and
wherein a second marking is included at a second position, corresponding to the second time interval, on the interactive computer-generated lifeline to represent the second aggregate.

16. The system of claim 15, wherein the processor when executed is further configured to:
display, in response to receiving a selection of the first marking, a listing of entries, where each entry is associated with a different record of the first set of records that make up the first aggregate; and
display, in response to receiving a selection of a particular entry, information associated with a particular record of the first set of records.

17. The system of claim 15, wherein the first marking is a first size and represents a first severity of harm, and the second marking is a second size and represents a second severity of harm that is different than the first severity of harm.

18. A system, comprising:
a processor when executed configured to:
import a plurality of records, for a subject, from a source and/or an end user device;
create, based on natural language processing and machine learning, an output record for each of the plurality of imported records, where each output record includes a body system identifier for an affected body system, a body part identifier for an affected body part, an impact identifier for an impact level, an is-active identifier for whether a condition is active or not, and a date identifier identifying a particular date;
create a first aggregate for a first set of the output records, wherein each output record of the first aggregate includes a first same body part identifier, assigned to a first body part, for the affected body part;
create a second aggregate for a second set of the output records, wherein each output record of the second aggregate includes a second same body part identifier, assigned to a second body part, for the affected body part;
generate an interactive computer-generated body map for the user, the interactive computer-generated body map including at least a front view of a human avatar and a back view of the human avatar, wherein the front view and the back view include a pictorial representation of a human anatomy having body parts;
highlight the first body part on the interactive computer-generated body map with a first graphical affordance indicating a first severity of harm, wherein the first severity of harm for the first body part is determined based on at least the impact identifier for the impact level included in the first set of records that make up the first aggregate; and
highlight the second body part on the interactive computer-generated body map with a second graphical affordance indicating a second severity of harm, wherein the second severity of harm for the second body part is determined based on at least the impact identifier included in the second set of output records that make up the second aggregate.

19. The system of claim 18, wherein the processor when executed is further configured to:
display, in response to receiving a selection of the first body part, a listing of entries associated with the first set of output records that make up the first aggregate; and
display, in response to receiving a selection of a particular entry form the listing, information associated with a particular record of the first set of records.

20. The system of claim 18, wherein the records include medical records.
